(19) United States
(12) Patent Application Publication
(10) Pub. No.: US 2018/0078970 A1
(43) Pub. Date: Mar. 22, 2018

(54) PIEZOELECTRIC DEVICE AND ULTRASONIC APPARATUS

(71) Applicant: Kabushiki Kaisha Toshiba, Minato-ku (JP)

(72) Inventors: Tomio ONO, Yokohama (JP); Kazuhiro ITSUMI, Koto (JP); Kazuhiko HIGUCHI, Kawasaki (JP)

(73) Assignee: Kabushiki Kaisha Toshiba, Minato-ku (JP)

(21) Appl. No.: 15/437,963

(22) Filed: Feb. 21, 2017

(30) Foreign Application Priority Data
Sep. 16, 2016 (JP) 2016-181987

(51) Int. Cl. B06B 1/06; H01L 41/053; H01L 41/047; A61B 8/00

(52) U.S. Cl. CPC B06B 1/0629; H01L 41/0533; A61B 8/4272; H01L 41/0475; A61B 8/4483; B06B 1/0603

(57) ABSTRACT

A piezoelectric device according to an embodiment comprises a piezoelectric thin film, a first electrode disposed on a first surface of the piezoelectric thin film, a substrate provided with an electrode pad, a plurality of pillar-shaped first supporting members provided between a second surface on an opposite side of the first surface of the piezoelectric thin film and the electrode pad of the substrate so as to fix the piezoelectric thin film onto the substrate, and a plurality of second electrodes electrically connected to the electrode pad from a part of the second surface of the piezoelectric thin film via a lateral surface of the first supporting member. The piezoelectric thin film, the first electrode, and the second electrodes compose a plurality of diaphragms each of which is a transducer element. The first supporting members are provided at locations at which the respective diaphragms are sectioned. The first electrode is provided in common to the diaphragms.

### CROSS-REFERENCE TO RELATED APPLICATIONS

This application is booed upon and claims the benefit of priority from Japanese Patent Application No. 2016-181987, filed on Sep. 16, 2016; the entire contents of which are incorporated herein by reference.

### FIELD

Embodiments described herein relate generally to a piezoelectric device and an ultrasonic apparatus.

### BACKGROUND

In recent years, piezoelectric thin-film manufacturing technologies have been improved, and the application of piezoelectric thin-film devices to sensors and actuators has been explored. That includes an ultrasonic diagnostic imaging apparatus for medical use and an ultrasonic inspection device for non-destructive inspection. Those are apparatuses that acquire internal information of a subject, by transmitting ultrasound to the subject from an ultrasonic probe and receiving with the probe the ultrasound that is reflected in the inside of the subject.

A conventional ultrasonic probe has a configuration that piezoelectric transducer elements composed of piezoelectric ceramic such as lead zirconate titanate (PZT) are one-dimensionally or two-dimensionally arrayed. In the following description, each transducer element is referred to as an element. In such a configuration, giving different delays to transmission pulse signals provided to the respective elements in transmission makes it possible to perform deflection and convergence of an ultrasonic beam. Similarly, also in receiving, giving different delays to receiving pulse signals obtained by the respective elements and summing them make it possible to emphasize and receive a signal of an intended direction and distance. These manipulations of ultrasonic beam are referred to as beam forming.

### BRIEF DESCRIPTION OF THE DRAWINGS

FIG. 1 is a sectional elevation illustrating an example of a schematic configuration of a piezoelectric device according to a first embodiment;

FIG. 2 is a cross-sectional view taken along the line A-A of the piezoelectric device illustrated in FIG. 1;

FIG. 3 is a diagram illustrating the deformation of a piezoelectric thin film when a voltage is applied to a pMUT element array in the first embodiment;

FIG. 4 is a sectional elevation of process illustrating one example of a manufacturing process of the piezoelectric device in the first embodiment (Part 1);

FIG. 5 is a sectional elevation of process illustrating one example of the manufacturing process of the piezoelectric device in the first embodiment (Part 2);

FIG. 6 is a sectional elevation of process illustrating one example of the manufacturing process of the piezoelectric device in the first embodiment (Part 3);

FIG. 7 is a sectional elevation illustrating an example of a schematic configuration of an ultrasonic probe in the first embodiment;

FIG. 8 is a cross-sectional view taken along the line B-B of the ultrasonic probe illustrated in FIG. 7;

FIG. 9 is a sectional elevation illustrating an example of a schematic configuration of an ultrasonic probe according to a second embodiment;

FIG. 10 is a sectional elevation illustrating an example of a schematic configuration of a piezoelectric device according to a third embodiment;

FIG. 11 is a cross-sectional view taker: along the line C-C of the piezoelectric device illustrated in FIG. 10;

FIG. 12 is a diagram illustrating the deformation of a piezoelectric thin film when a voltage is applied to a pMUT element array in the third embodiment;

FIG. 13 is a sectional elevation illustrating an example of a schematic configuration of an ultrasonic probe in the third embodiment;

FIG. 14 is a cress-sectional view taken along the line D-D of the ultrasonic probe illustrated in FIG. 13;

FIG. 15 is a sectional elevation illustrating an example of a schematic configuration of an ultrasonic probe according to a fourth embodiment;

FIG. 16 is a cross-sectional view taken along the line E-E of the ultrasonic probe illustrated in FIG. 15;

FIG. 17 is a graph illustrating frequency spectra of resonance frequencies of respective pMUT elements of the ultrasonic probe illustrated in FIGS. 15 and 16;

FIG. 18 is a graph illustrating a frequency spectrum that the frequency spectra illustrated in FIG. 17 were combined;

FIG. 19 is a sectional elevation illustrating an example of a schematic configuration of a piezoelectric device according to a fifth embodiment;

FIG. 20 is a cross-sectional view taken along the line F-F of the piezoelectric device illustrated in FIG. 13;

FIG. 21 is a sectional elevation illustrating an example of a schematic configuration of a piezoelectric device according to a sixth embodiment;

FIG. 22 is a cross-sectional view taken along the line G-G of the piezoelectric device illustrated in FIG. 21;

FIG. 23 is a sectional elevation illustrating an example of a schematic configuration of an ultrasonic apparatus according to a seventh embodiment;

FIG. 24 is a cross-sectional view taken along the line H-H of the ultrasonic apparatus illustrated in FIG. 23;

FIG. 25 is a sectional elevation illustrating an example of a schematic configuration of an ultrasonic apparatus according to an eighth embodiment;

FIG. 26 is a sectional elevation illustrating an example of a schematic configuration of an ultrasonic apparatus according to a ninth embodiment;

FIG. 27 is a block diagram illustrating an example of a schematic configuration of an ultrasonic probe according to a tenth embodiment;

FIG. 28 is a block diagram illustrating another example of the configuration of a piezoelectric device of the ultrasonic probe in the tenth embodiment;

FIG. 29 is a block diagram illustrating yet another example of the configuration of the piezoelectric device of the ultrasonic probe in the tenth embodiment;

FIG. 30 is a block diagram illustrating an example of a schematic configuration of an ultrasonic diagnostic apparatus according to an eleventh embodiment;

FIG. 31 is a block diagram illustrating an example of a schematic configuration of a transmitting and receiving unit of the ultrasonic diagnostic apparatus in the eleventh embodiment;

FIG. 32 is a diagram illustrating an example of a schematic configuration of a piezoelectric device used in explanation of a twelfth embodiment;

FIG. 33 is a graph illustrating a simulation result according to the twelfth embodiment;

FIG. 34 is a diagram illustrating an example of a schematic configuration of a piezoelectric device in a first comparative example used in the explanation of the twelfth embodiment;

FIG. 35 is a diagram illustrating an example of a schematic configuration of a piezoelectric device in a second comparative example used in the explanation of the twelfth embodiment;

FIG. 36 is a graph illustrating one example of area usage efficiencies calculated in the twelfth embodiment.

### DETAILED DESCRIPTION

With reference to the accompanying drawings, the following describes in detail piezoelectric devices and ultrasonic apparatuses according to exemplary embodiments.

In order to perform beam forming in an ultrasonic inspection apparatus and the like, the pitch of a single element needs to be smaller than λ/2, when the wavelength of the ultrasound is defined as λ. For example, in water, when the frequency of the ultrasound is defined as 3 megahertz (MHz), the wavelength of the ultrasound needs to be smaller than 250 μm.

When manufacturing an ultrasonic probe that has a large field of view, it only needs to make the size of the ultrasonic probe large. However, in the pitch of the element, there is the foregoing restriction. Thus, when the frequency is assumed to be constant, the number of elements of the probe is to increase in proportion to the size of the probe in the case of one-dimensional probe, and in proportion to the square of the size of the probe in the case of two-dimensional probe. In order to perform bear forming, a transmitting and receiving circuit (hereinafter referred to as a channel) is needed for each element, but it makes difficult to establish electrical connection of the elements and the channels because the number of channels also increases when the number of elements increases.

When manufacturing a probe that operates at a high frequency in order to increase resolution, because the wavelength of the ultrasound is shortened, it needs to make the pitch of the element small. Thus, when the size of the probe is assumed to be constant, the number of elements increases after all, and the same problem as that in the foregoing case of making the size large is to arise. Moreover, in this case, because the size of the element is made small, the manufacturing by the method of fabricating the elements by machining the piezoelectric ceramic is difficult.

As a way to resolve such problems of the foregoing, if is conceivable to use piezoelectric micromachined ultrasound transducers (pMUT) that utilize piezoelectric thin films and semiconductor microfabrication technologies.

The center frequency of a pMUT element is a mechanical resonance frequency of its diaphragm (equivalent to the pMUT element that is a single transducer element) that is determined by the thickness and the size of the diaphragm. Thus, the size of the diaphragm requires high accuracy. In order to form dense pMUT of a high area usage efficiency, it also needs to make the width of a partition wall as small as possible. This means that a high accuracy is required in deep reactive ion etching (RIE), in order to uniformly form dense and microscopic diaphragms in a wafer surface.

Furthermore, in pMUT, because an ultrasonic beam is formed in the tipper direction, a circuit board needs to be disposed on the lower side of the pMUT. Thus, out of two electrodes to apply a voltage to the pMUT, in order to connect an electrode that is not arranged on the circuit board side to the circuit board for each pMUT element, it needs to use a penetration structure such as a through-silicon via (TSV). Accordingly, the usage efficiency of area is to be decreased for the area of the penetration structure.

As in the foregoing, in the structure that fixes the end portions of the diaphragm by the partition walls, due to the occupied area of the partition walls, there has been a problem in that the area usage efficiency of the pMUT is deteriorated. In the structure that uses a penetration structure such as TSV for electrically connecting the pMUT and the circuit board, there has been a problem in that, due to the occupied space of the penetration structure, the area usage efficiency of the pMUT is further deteriorated.

Thus, in the following embodiments, a piezoelectric device and an ultrasonic apparatus for which the area usage efficiency has been improved will be described with examples given. Some of the embodiments exemplified in the following further have an effect in that the manufacturing is possible by a simple manufacturing method. Some of the embodiments exemplified in the following further have an effect in that it is possible to reduce the lowering of sensitivity due to a parasitic capacitance.

### First Embodiment

With reference to the accompanying drawings, the following describes in detail a piezoelectric device and an ultrasonic apparatus according to a first embodiment.

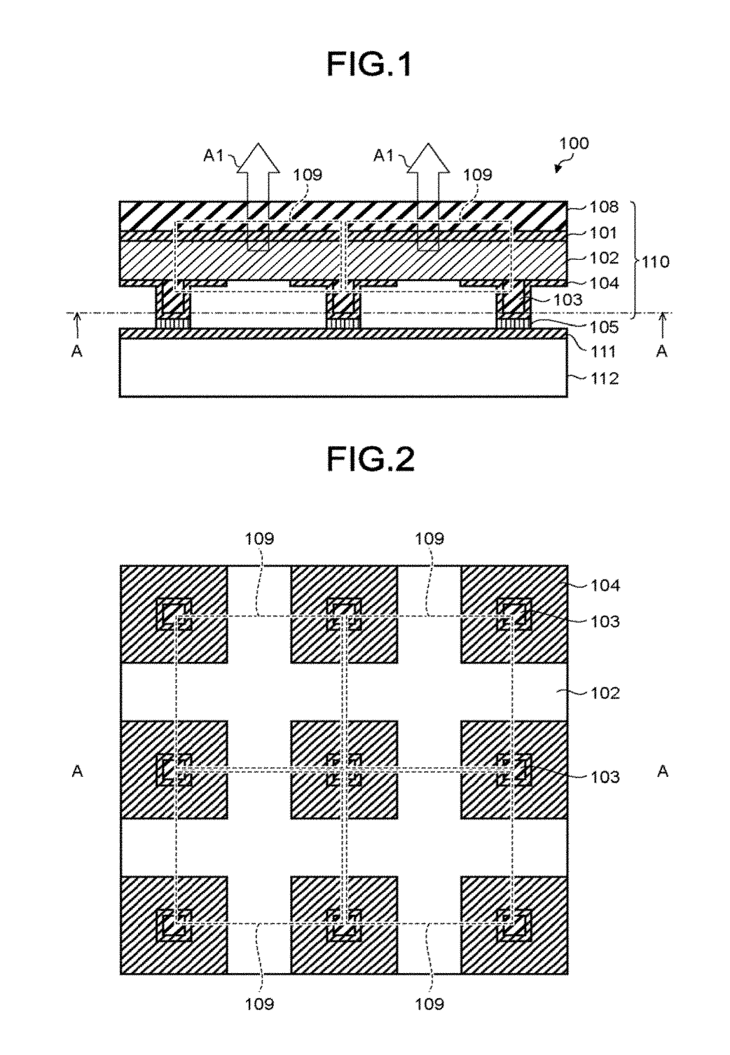

FIG. 1 is a sectional elevation illustrating an example of a schematic configuration of a piezoelectric device in the first embodiment, and FIG. 2 is a cross-sectional view taken along the line A-A of the piezoelectric device illustrated in FIG. 1. FIG. 1 illustrates a cross section structure of a plane perpendicular to a pMUT mounting surface of a circuit board 112.

As illustrated in FIGS. 1 and 2, a piezoelectric device 100 in the first embodiment includes a pMUT element array 110, and the circuit board 112 that includes an electrode pad 111.

The pMUT element array 110 includes a piezoelectric thin film 102, a first electrode 101, a plurality of supporting members 103, a plurality of second electrodes 104, and a support layer 108. In the following description, a structure that is structured with the piezoelectric thin film 102, the first electrode 101, and the second electrodes 104 that correspond to art area surrounded by four pieces of the supporting members 103 that are vertically and horizontally adjacent to one another in this configuration is referred to as a diaphragm 109. It is further assumed that a single diaphragm 109 corresponds to a single pMUT element that is a single transducer element (unit).

The piezoelectric thin film 102 is a member that vibrates in accordance with a voltage applied between the first electrode 101 and the second electrode 104. For this piezoelectric thin film 102, a piezoelectric material such as aluminum nitride (AlN), zinc oxide (ZnO), lead zirconate titanate (PZT), lead titanate (PbTiO₃), lead zirconate (PbZrO₃), barium titanate barium strontium titanate, and lead lanthanum titanate ((Pb,La)TiO₃) can be used, for example.

The first electrode 101 is an electrode common to a plurality of diaphragms 109 and is extending on a first surface of the piezoelectric thin film 102 so as to extend over the plurality of diaphragms 109. The first electrode 101 may be grounded. For such a first electrode 101, a metal or an alloy such as aluminum (Al), silver (Ag), gold (Au), titanium (Ti), tungsten (W), and nickel (Ni) can be used, for example.

Each supporting member 103 is a pillar member for which the cross section has a square, hexagonal, or rotund shape. However, the shape of the supporting member 103 is not limited to the shape of a square pillar, hexagonal column, circular column, or the like. For example, it may be in a trapezoidal or spherical shape, or even in a squashed shape of the foregoing. In the following description, the shape including the foregoing shapes is referred to as a pillar shape.

For the supporting member 103, an insulation material such as silicon oxide (SiO₂) can be used, for example. As illustrated in FIG. 2, on a second surface that is the opposite side of the first surface of the piezoelectric thin film 102, a plurality of supporting members 103 are periodically disposed at a certain distance. Accordingly, the diaphragms 109 also form a periodic array. In other words, the supporting members 109 are provided at locations that section each of the diaphragms 109 (at four corners of the respective diaphragms 109 in FIG. 2). In FIG. 2, a total of four diaphragms 109 in two rows and two columns are illustrated. However, the number of diaphragms 109 (that is, the number of pMUT elements) in a single piezoelectric device 100 is not limited to four. That is, as long as the diaphragms 109 are configured in a periodic array, the number of pMUT elements may be changed as appropriate.

The second electrodes 104 are individually provided at the locations corresponding to the respective supporting members 103. Each of the second electrodes 104 is formed so as to extend at least from the second surface of the piezoelectric thin film 102 to the lateral surfaces of the supporting member 103. It is preferable that the area that the second electrode 104 and the second surface of the piezoelectric thin film 102 have contact be larger than the surface that the supporting member 103 and the second surface have contact and be an area in a degree of not making contact with the other adjacent second electrodes 104. Of both ends of the supporting member 103, on the end side that is the opposite side (this is referred to as a second end) to the end that has contact with the piezoelectric thin film 102 (this is referred to as a first end), the second electrode 104 is extending to a degree of facilitating the physical and electrical connection with a later-described adhesive layer 105. In the example illustrated in FIG. 1, the second electrode 104 is formed so as to cover the second end of the supporting member 103.

The second electrode 104 is what is called an operation electrode to which a drive voltage for operating the piezoelectric thin film 102 is applied. Accordingly, by providing wiring for electrically connecting the second electrode 104 to the circuit board 112 not on the surface of the piezoelectric thin film 102 but on the supporting member 103, it mates it possible to drastically reduce the parasitic capacitance. For the second electrode 104, as with the first electrode 101, a metal or an alloy such as aluminum (Al), silver (Ag), gold (Au), titanium (Ti), tungsten (W), and nickel (Ni) can be used, for example.

The support, layer 108 is a layer that serves as a base when forming such a layer structure as in the foregoing. in the first embodiment, as the support layer 108, a silicon layer is illustrated, and the thickness thereof is defined as hₚ.

The pMUT element array 110 thus configured is bonded onto the electrode pad ill of the circuit board 112 that is a base substrate, by using the adhesive layer 105. Consequently, the pMUT element array 110 is mechanically fired onto the circuit board 112 and pMUT element array 110 is electrically connected to a drive circuit mounted on the circuit board 112. In place of the circuit board 112, a supporting substrate that includes only the electrode pad 111 and wiring may be used. In this case, the drive circuit that drives the pMUT elements is disposed outside the supporting substrate.

The circuit board 112 is constructed by using a silicon substrate, for example, and is equipped with a drive circuit that includes a transmitting circuit that drives to excite the piezoelectric thin film 102 and a receiving circuit that converts the vibration of the piezoelectric thin film 102 into an electrical signal.

For the adhesive layer 105 that bonds the second electrode 104 of the pMUT element array 110 and the electrode paid 111 of the circuit board 112, a conductive adhesive layer of such as germanium (Ge) cars be used. For the electrode pad 111, a metal or an alloy such as aluminum (Al), silver (Ag), gold (Au), titanium (Ti), tungsten (W), and nickel (Ni) can be used, for example.

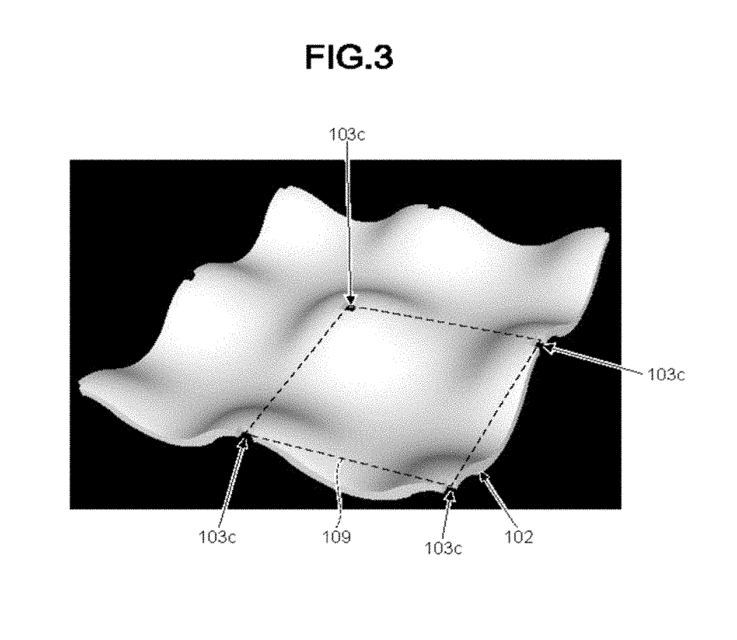

Next, the operation of the piezoelectric device 100 illustrated in FIGS. 1 and 2 will be described. FIG. 3 is a diagram illustrating the deformation of a piezoelectric thin film when a voltage is applied to the pMUT element array in the first embodiment. In FIG. 3, holes 103c correspond to the supporting members 103 of a pillar shape.

In the pMUT element array 110 in the first embodiment, the pMUT elements (the diaphragms 109) are not mechanically independent. Thus, the deformation interferes with each other between the adjacent pMUT elements. However, in the first embodiment, the second electrodes 104 that induce the piezoelectric effect are disposed having symmetry. Accordingly, as illustrated in FIG. 3, the piezoelectric thin film 102 of each pMUT element deforms in a barrel shape in the same manner as in the case of the individual pMUT elements (the diaphragms 109) being mechanically independent.

When the pMUT element array 110 is composed of, for example, a total of four pMUT elements in two rows and two columns, there is a vibration mode for which the resonance frequency is lower than a vibration mode in which all the four pMUT elements vibrate in the same phase. In the example illustrated in FIG. 3, in the drawing, a vibration mode in which the upper two pMUT elements and the lower two pMUT elements vibrate in reverse phase and others are present. However, in the first embodiment, because the second electrodes 104 are disposed in symmetry, such a vibration mode other than the vibration mode in which all the four pMUT elements vibrate in the same phase is suppressed, and thus a vibration mode other than a target resonance frequency is never excited.

From the foregoing, as illustrated in FIG. 3, the pMUT elements of the pMUT element array 110 in the first embodiment can vibrate in a vibration mode in which all those elements vibrate in the same phase. As a result, in the example illustrated in FIG. 3, an ultrasonic boom that is generated by the volume changes in which the four pMUT elements deform in a barrel shape in the same phase is emitted in the direction of arrows A1 in FIG. 1.

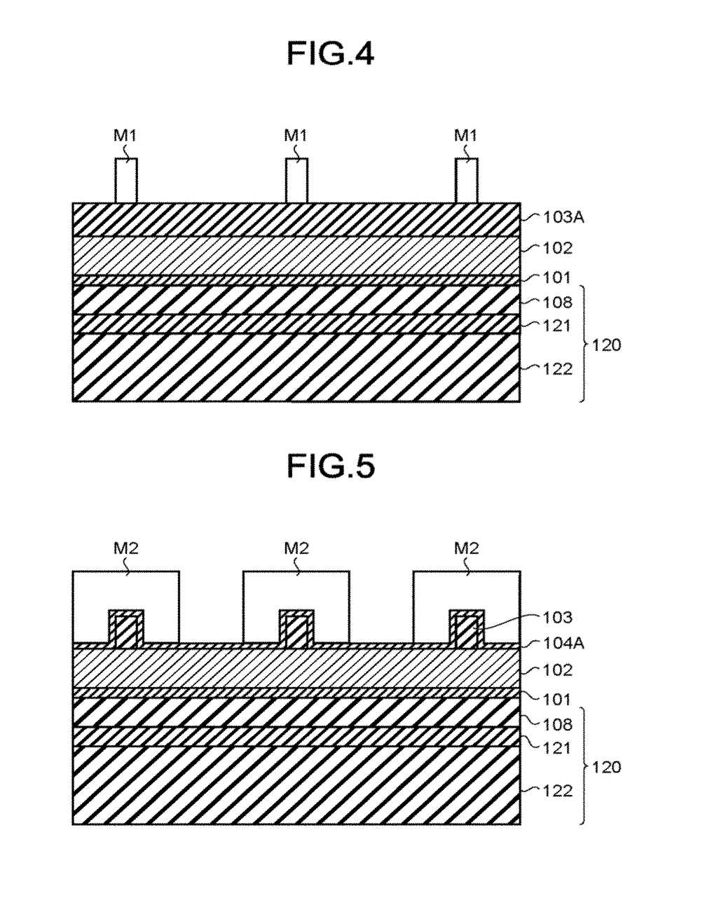

Next, with reference to the accompanying drawings, a manufacturing method of the piezoelectric device 100 in the first embodiment will be described in detail. FIGS. 4 to 6 are sectional elevations of process illustrating one example of the manufacturing process of the piezoelectric device in the first embodiment.

In this manufacturing method, as a base substrate, a silicon on insulator (SOI) substrate 120 that includes a buried oxide film 121 and a silicon layer (the support layer 108) on a silicon substrate 122 is used. Thus, in the following description, the support layer 108 in FIG. 1 will be described by substituting a silicon thin film 108 for it.

In this manufacturing method, as illustrated in FIG. 4, on the silicon thin film 108 of the SOI substrate 120, the first electrode 101, the piezoelectric thin film 102, and a silicon oxide film 103A are first formed in sequence. On the silicon oxide film 103A, a mash film M1 on which a pattern of the supporting members 103 is transferred is further formed. For the forming of the first electrode 101, the piezoelectric thin film 102, and the silicon oxide film 103A, a sputtering method, an epitaxial growth method, or the like can be used. For the mask film M1, material such as a silicon nitride film and others that has etch selectivity to the silicon oxide film 103A can be used. For the patterning, a patterning technique that utilizes a photolithographic technique and an etching technique can be used.

Then, by etching the silicon oxide film 103A by using the mask film M1 as a mask, the silicon oxide film 103A is made into the supporting members 103. For the etching of the silicon oxide film 103A, dry etching such as reactive ion etching (RIE) can be used, for example. Subsequently, on the piezoelectric thin film 102 on which the supporting members 103 have been formed, a conductive film 104A to be made into the second electrodes 104 is formed. For the forming of the conductive film 104A, a sputtering method, an epitaxial growth method, or the like can be used. Then, as illustrated in FIG. 5, on the conductive film 104A, a mask film M2 on which a pattern of the second electrodes 104 is transferred is formed. For the mask film M2, a resist film can be used. For the patterning thereof, a patterning technique that utilizes a photolithographic technique can be used.

Then, by etching the conductive film 104A by using the mask film M2 as a mask, the conductive film 104A is made into the second electrodes 104. Accordingly, on the silicon thin film 108 of the SOI substrate 120, pMUT elements are formed. For the etching of the conductive film 104A, wet etching that uses a certain etchant and dry etching can be used, for example.

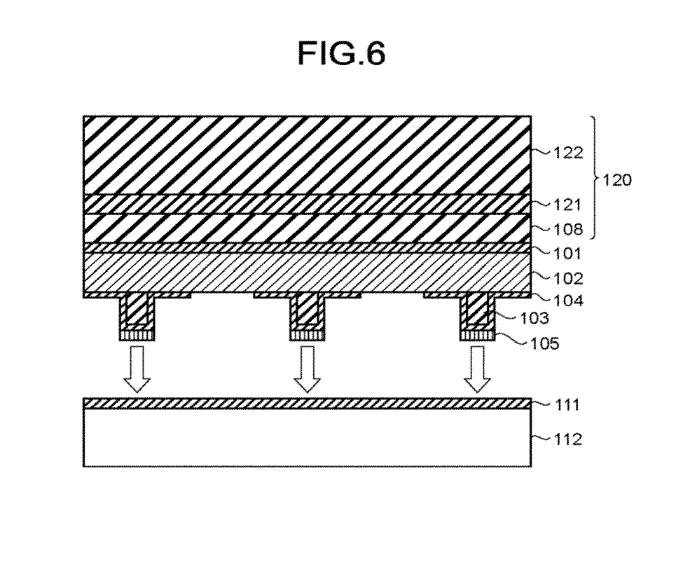

Then, on the second electrodes 104 on the supporting members 103, the adhesive layer 105 is formed. In this description, the material used for the adhesive layer 105 is assumed to be germanium (Ge). For the forming of the adhesive layer 105, a lift-off method and the like can be used, for example. Then, as illustrated in FIG. 6, the SOI substrate 120 on which the adhesive layer 105 has been formed is turned upside down, and the SOI substrate 120, con which the pMUT elements have been formed, and the circuit board 112 are bonded while performing the positioning of the adhesive layer 105 and the electrode pad 111. In this description, the adhesive layer 105 is made of germanium (Ge), and the second electrodes 104 and the electrode pad 111 are made of aluminum (Al). In that case, because an Al—Ge eutectic bonding is formed by heating in the atmosphere, for the bonding of the SOI substrate 120, on which the pMUT elements are formed, and the circuit board 112, a heating process in the atmosphere can be used.

Thereafter, by etching the buried oxide film 121 of the SOI substrate 120, the buried oxide film 121 and the silicon substrate 122 are removed from the silicon thin film 108. Accordingly, the piezoelectric device 100 of a layer structure illustrated in FIG. 1 is manufactured.

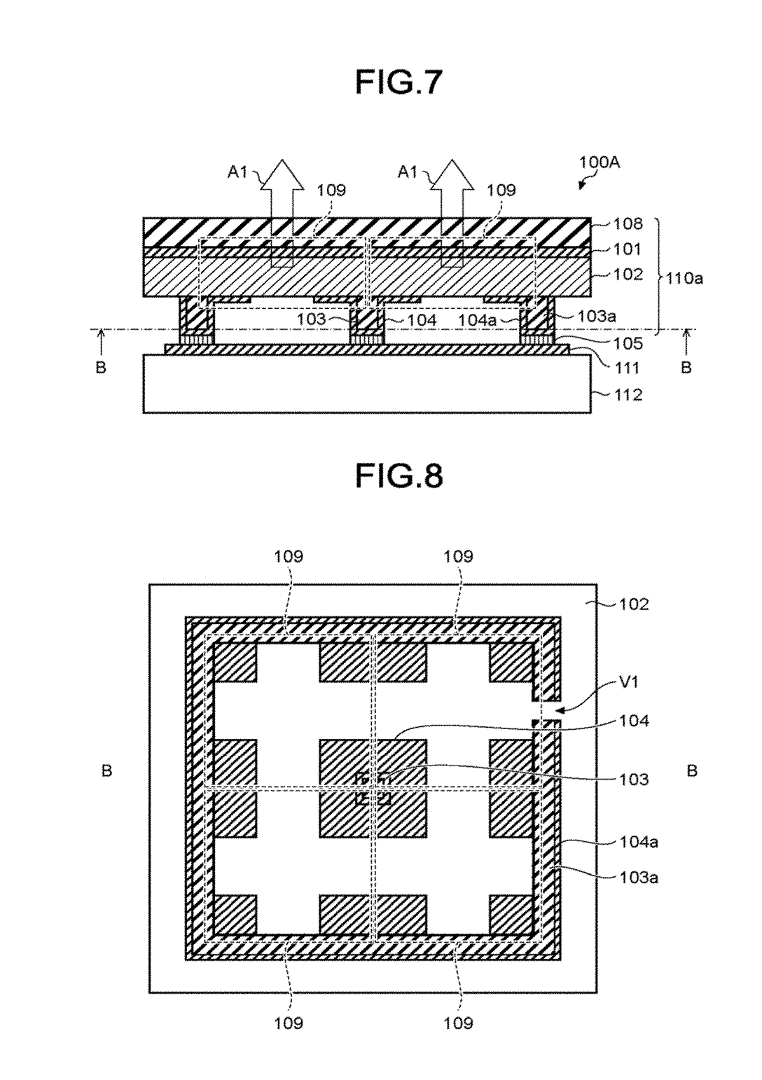

Next, with reference to the accompanying drawings, an ultrasonic apparatus that uses the piezoelectric device 100 in the first embodiment as a single transducer element group (hereinafter referred to as an element) will be described in detail. In the following description, an ultrasonic probe is exemplified as the ultrasonic apparatus. FIG. 7 is a sectional elevation illustrating an example of a schematic configuration of the ultrasonic probe in the first embodiment, and FIG. 8 is a cross-sectional view taken along the line B-B of the ultrasonic probe illustrated in FIG. 7. FIG. 7 illustrates, as with that of FIG. 1, a cross section structure of a plane perpendicular to a pMUT mounting surface of the circuit board 112. In this description, an ultrasonic probe 100A is assumed to include a plurality of elements. In this description, illustrated is the case that each element includes four pMUT elements in two rows and two columns. However, as with that of the foregoing, as long as the diaphragms 109 are configured in a periodic array, the number of pMUT elements may be changed as appropriate.

As illustrated in FIGS. 7 and 8, the piezoelectric device in the ultrasonic probe 100A includes the same configuration as that of the piezoelectric device 100 illustrated in FIGS. 1 and 2. However, in the piezoelectric device in the ultrasonic probe 100A, of the supporting members 103 arrayed in three rows and three columns in the piezoelectric device 100, the supporting members 103 other titan the center supporting member 103 are replaced with a fence-like supporting member 103a that surrounds the center supporting member 103, and the second electrodes 104 disposed on the supporting members 103 before replacing are replaced with a second electrode 104a that is disposed on the replaced supporting member 103a.

The second electrode 104a is formed so as to extend at least from the second surface of the piezoelectric thin film 102 to the lateral surfaces of the supporting member 103a. The second electrode 104a is extending toward the second end side of the supporting member 103a to a degree of facilitating the physical and electrical connection with the adhesive layer 105. In the example illustrated in FIG. 7, the second electrode 104a is formed so as to cover the second end of the supporting member 103a.

As illustrated in FIGS. 7 and 8, when the piezoelectric device 100 is used as an element of the ultrasonic probe 100A, there is a need to decouple the mechanical coupling between elements. The methods of decoupling the mechanical coupling between elements include a way to fix the individual diaphragms 109 or a way to physically separate the diaphragms 109 between elements.

However, when the ultrasonic probe 100A is used, because the ultrasonic probe 100A contacts a test subject via acoustic coupling material having fluidity, in the configuration that the diaphragms 109 are physically separated, there is a possibility that the acoustic coupling material invades the inside of the diaphragms 109. Thus, in the example illustrated in FIGS. 7 and 8, a configuration that is capable of preventing the acoustic coupling material from invading and fixes individual elements (pMUT element arrays 110a) is employed. In this example, the configuration to fix the individual elements (the pMUT element arrays 110a) corresponds to the supporting member 103a. When the individual elements (the pMUT element arrays 110a) are fixed, the periodicity of the pMUT is disturbed. However, even when the surround of the pMUT element array 110a is fixed or severed, as illustrated in FIG. 3, the piezoelectric thin film 102 of each pMUT element deforms in a barrel shape.

On the fence-like supporting member 103a provided so as to surround the center supporting member 103, a communication path V1 is provided so that a hermetically closed space is not formed between the piezoelectric thin film 102 and the circuit board 112. Accordingly, it can be reduced that the deformation of the piezoelectric thin film 102 is hindered by the pressure of gas sealed in the hermetically closed space. Furthermore, when it is not a configuration that individual pMUT elements are fixed by the supporting members 103 but a configuration that it is fixed by units of an element, the number of communication paths V1 provided on the supporting member 103a can be small, and thus the complexity in manufacturing process can be reduced. To prevent the acoustic coupling material from invading from the communication path V1, it is preferable that the opening sire of the communication path V1 be small to an extent necessary and sufficient. The communication path V1 may be communicating with the outside air, or communicating with other elements.

Moreover, in the example illustrated in FIGS. 7 and 8, the number of pMUT element, arrays 110a of each element is a total of four in two rows and two columns. However, by increasing the number of pMUT element arrays 110a of each element, the rate of the area that the supporting member 103 and the supporting member 103a occupy in each element can be reduced. As a result, the usage efficiency of area that contributes to the generation of the ultrasound (hereinafter referred to as an area usage efficiency) can be made high. The area usage efficiency can be expressed by the rate of the area of the portion that contributes to the generation of the ultrasound, out of the area of the first surface (the second surface) of the piezoelectric thin film 102, for example. The portion that contributes to the generation of the ultrasound can be defined as a portion that deforms in the piezoelectric thin film 102. In order to increase this deforming portion, it is also important to make the portions of the second electrode 104 and the second electrode 104a, which contact to the piezoelectric thin film 102, small.

As in the foregoing, because the structure of the pMUT elements in the first embodiment is a configuration that partition walls are not provided for each pMUT element, the area usage efficiency can be made high. Accordingly, the efficient generation of the ultrasonic beam is made possible.

Furthermore, in the structure of the pMUT elements in the first embodiment, because a hermetically closed space is not formed by the partition walls, it can be reduced that the deformation of the piezoelectric thin film 102 is hindered by the pressure of gas sealed in the hermetically closed space. Accordingly, because the piezoelectric thin film 100 can be made to efficiently deform, more efficient generation of the ultrasonic beam is made possible.

According to the first embodiment, because the second electrodes 104 (and 104a) are provided at the locations corresponding to the supporting members 103 (and 103a), it makes it possible to electrically connect the second electrodes 104 (and 104a) to the electrode pad 111 disposed on the circuit board 112 easily. The structure of the pMUT elements in the first embodiment is a structure that is capable of reducing or omitting auxiliary electrodes that connect among the first electrode 101, the second electrodes 104, and the electrode pad 111. By such a configuration, because a parasitic capacitance between the electrodes can be reduced, the piezoelectric thin film 102 can be mace to efficiently deform with respect to an applied voltage. As a result, more efficient generation of the ultrasonic beam is made possible.

According to the first embodiment, because a branding process in vacuum is not needed for the bonding of the second electrodes 104 (and 104a) and the electrode pad 111, it is possible to facilitate the manufacturing process.

### Second Embodiment

Next, with reference to the accompanying drawings, the following describes in detail a piezoelectric device and an ultrasonic apparatus according to a second embodiment. In the following description, the configurations the same as those described in the foregoing first embodiment are given the identical reference signs and the redundant explanations thereof are omitted.

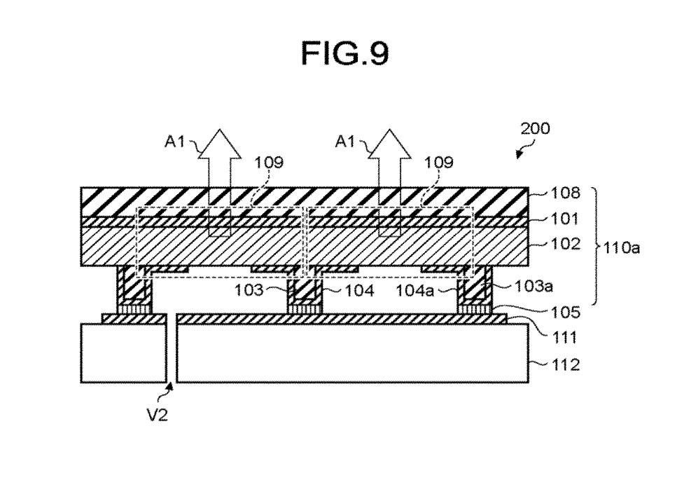

FIG. 9 is a sectional elevation illustrating an example of a schematic configuration of an ultrasonic probe in the second embodiment. FIG. 9 illustrates a cross section structure of a plane perpendicular to the pMUT mounting surface of the circuit board 112.

As illustrated in FIG. 9, an ultrasonic probe 200A in the second embodiment is provided with a configuration that, in the configuration the same as that of the ultrasonic probe 100A in the first embodiment (see FIGS. 7 and 8), a communication path V2 that runs through to the electrode pad 111 from the back of the circuit board 112 is provided on the circuit board 112. The communication path V2 is, as with the communication path V1 in the ultrasonic probe 100A, a hole for preventing a hermetically closed space from being formed between the piezoelectric thin film 102 and the circuit board 112.

Accordingly, also by providing the communication path V2 that runs through to the electrode pad 111 from the back of the circuit board 112, as with that of the first embodiment, it can be reduced that the deformation of the piezoelectric thin film 102 is hindered by the pressure of gas sealed in the hermetically closed space. In the second embodiment, because the communication path V2 never makes contact with the acoustic coupling material, the restriction for the opening size of the communication path V2 can be virtually eliminated.

The communication path V2 that runs through the circuit board 112 can be formed by using deep RIE that is a substrate penetration technique, for example.

In the second embodiment, the communication path V1 formed on the supporting member 103a of the ultrasonic probe 100A in the first embodiment may be omitted. In that case, because the process to provide the supporting member 103a with the communication path V1 can be omitted, it makes it possible to facilitate the manufacturing process.

Other configurations, operations, and effects can be the same as the configurations, operations, and effects in the first embodiment, and thus the redundant explanations are omitted.

### Third Embodiment

Next, with reference to the accompanying drawings, the following describes in detail a piezoelectric device and an ultrasonic apparatus according to a third embodiment. In the following description, the configurations the same as those described in the foregoing embodiments are given the identical reference signs and the redundant explanations thereof are omitted.

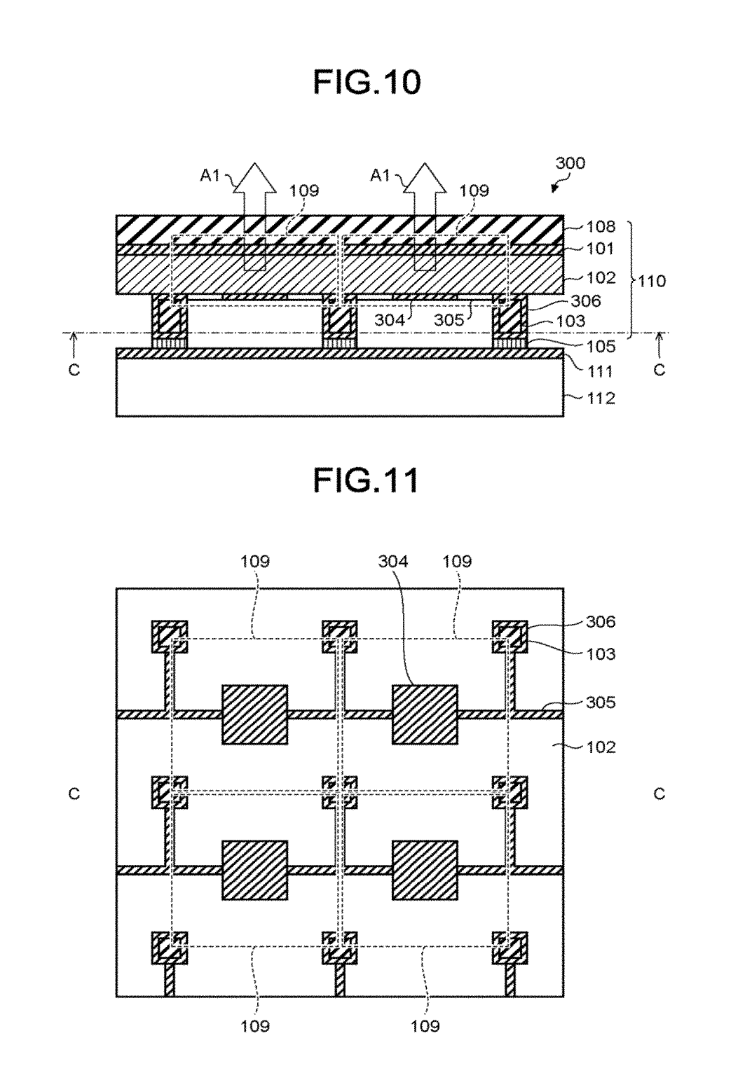

FIG. 10 is a sectional elevation illustrating an example of a schematic configuration of the piezoelectric device in the third embodiment, and FIG. 11 is a cross-sectional view taken along the line C-C of the piezoelectric device illustrated in FIG. 10. FIG. 10 illustrates a cross section structure of a plane perpendicular to the pMUT mounting surface of the circuit board 112.

As illustrated in FIGS. 10 and 11, a piezoelectric device 300 in the third embodiment is provided with a configuration that, in the same configuration as that of the piezoelectric device 100 in the first embodiment (see FIGS. 1 and 2), the second electrodes 104 are replaced with second electrodes 304, first auxiliary electrodes 305, and second auxiliary electrodes 306.

In the third embodiment, the second electrode 304 is disposed on substantially the middle of each of the diaphragms 109 on the second surface of the piezoelectric thin film 102. On the lateral surfaces of each supporting member 103, the second auxiliary electrode 306 that physically and electrically connects with the adhesive layer 105 is disposed on the second end side of the supporting member 103. In the example illustrated in FIG. 10, the second auxiliary electrode 306 is formed so as to cover the second end of the supporting member 103. Each second electrode 304 is electrically drawn around to the supporting member 103 by the first auxiliary electrode 305 formed on the second surface of the piezoelectric thin film 102, and is electrically connected to the second auxiliary electrode 306.

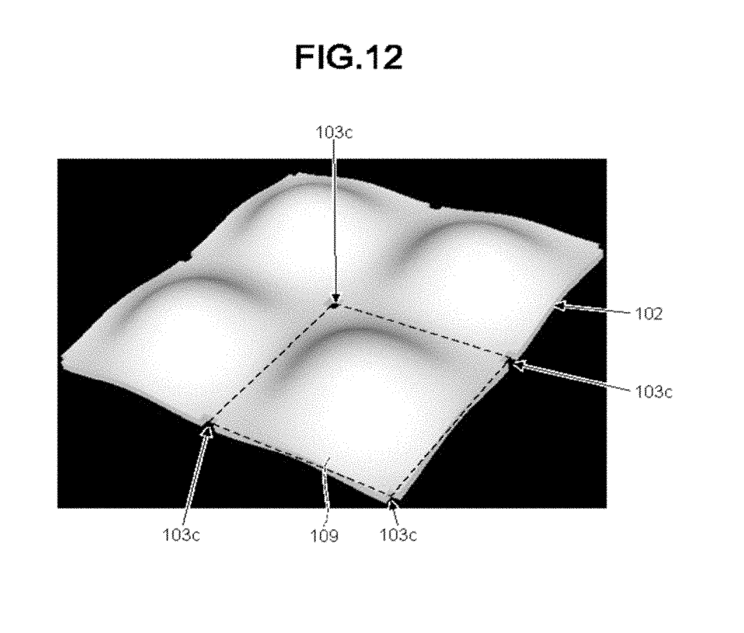

Next, the operation of the piezoelectric device 300 illustrated in FIGS. 10 and 11 will be described. FIG. 12 is a diagram illustrating the deformation of a piezoelectric thin film when a voltage is applied to the pMUT element array in the third embodiment. In FIG. 12, the holes 103c correspond to the supporting members 103 of a pillar shape.

As illustrated in FIG. 12, even when the second electrode 304 that is an operation electrode is provided not at the fixing portions of the pMUT element (the supporting member 103 portions) but in the central portion of each diaphragm 109, as with the piezoelectric device 100 in the first embodiment illustrated in FIG. 3, the piezoelectric thin film 102 deforms in a barrel shape and an unnecessary vibration mode is not excited.

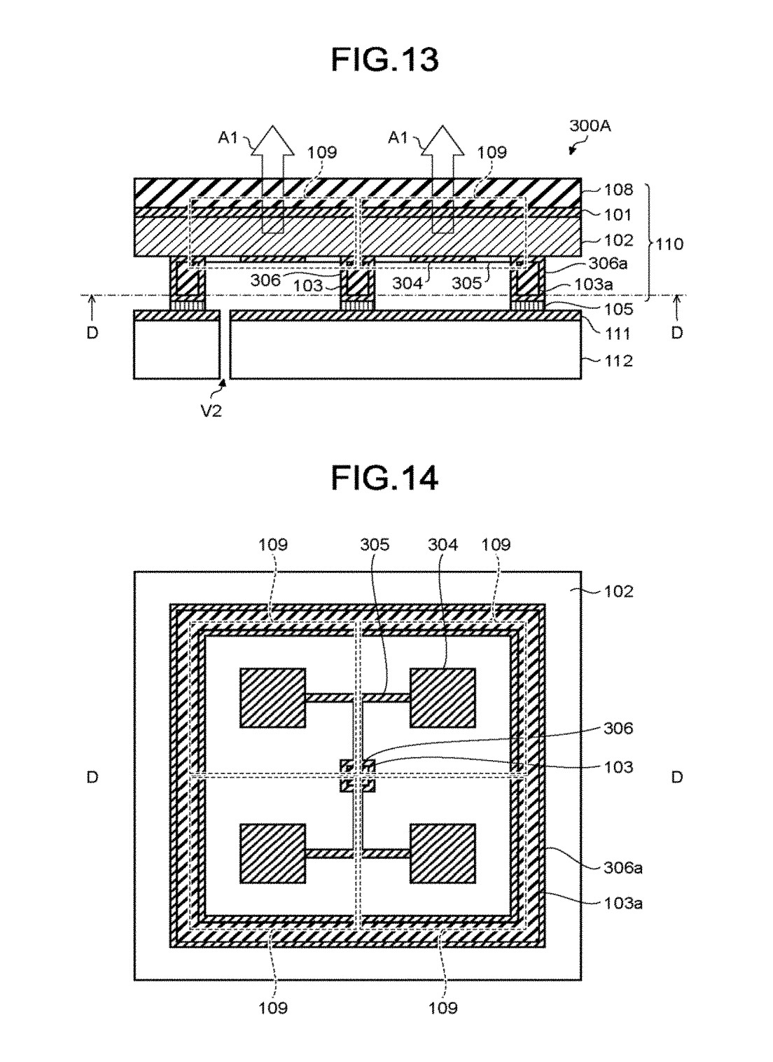

Then, with reference to the accompanying drawings, an ultrasonic probe that uses the piezoelectric device 300 in the third embodiment as an element will be described in detail. FIG. 13 is a sectional elevation illustrating an example of a schematic configuration of the ultrasonic probe in the third embodiment, and FIG. 14 is a cross-sectional view taken along the line D-D of the ultrasonic probe illustrated in FIG. 13. FIG. 13 illustrates, as with FIG. 10, a cross section structure of a plane perpendicular to a pMUT mounting surface of the circuit board 112. In this description, an ultrasonic probe 300A is assumed to include a plurality of elements. In this description, illustrated is the case that each element includes four pMUT elements in two rows and two columns. However, as with those of the foregoing, as long as the diaphragms 109 are configured in a periodic array, the number of pMUT elements may be changed as appropriate.

As illustrated in FIGS. 13 and 14, the piezoelectric device in the ultrasonic probe 300A has a configuration that the same nod if location as that of the ultrasonic probe 100A that is illustrated in FIGS. 7 and 8 has been added to the same configuration as that of the piezoelectric device 300 that is illustrated in FIGS. 10 and 11. However, in the configuration illustrated in FIGS. 13 and 14, the communication path V1 provided on the supporting member 103a has been replaced with the communication path V2 provided on the circuit board 112. The second electrode 301 disposed on the middle of each diaphragm 109 is electrically connected via the first auxiliary electrode 305 to the second auxiliary electrode 300 that is disposed on the center supporting member 103.

In the third embodiment, although the parasitic capacitance is somewhat increased cue to the first auxiliary electrode 305, when second auxiliary electrode 300a that electrically connects to the second electrodes 304 is converged to the second auxiliary electrode 306 disposed on the center supporting member 103, it is possible to omit the second auxiliary electrode 300a formed on the supporting member 103a. In that case, because the parasitic capacitance at the supporting member 103a portion can be reduced, it is possible, as a result, to reduce the effects due to the auxiliary electrode.

Other configurations, operations, and effects can be the same as the configurations, operations, and effects in the above-described embodiments, and thus the redundant explanations are omitted.

### Fourth Embodiment

In a fourth embodiment, modifications of the piezoelectric device and the ultrasonic apparatus in the above-described embodiments will be described. In the following description, the configurations the same as those described in the foregoing embodiments are given the identical reference signs and the redundant explanations thereof are omitted.

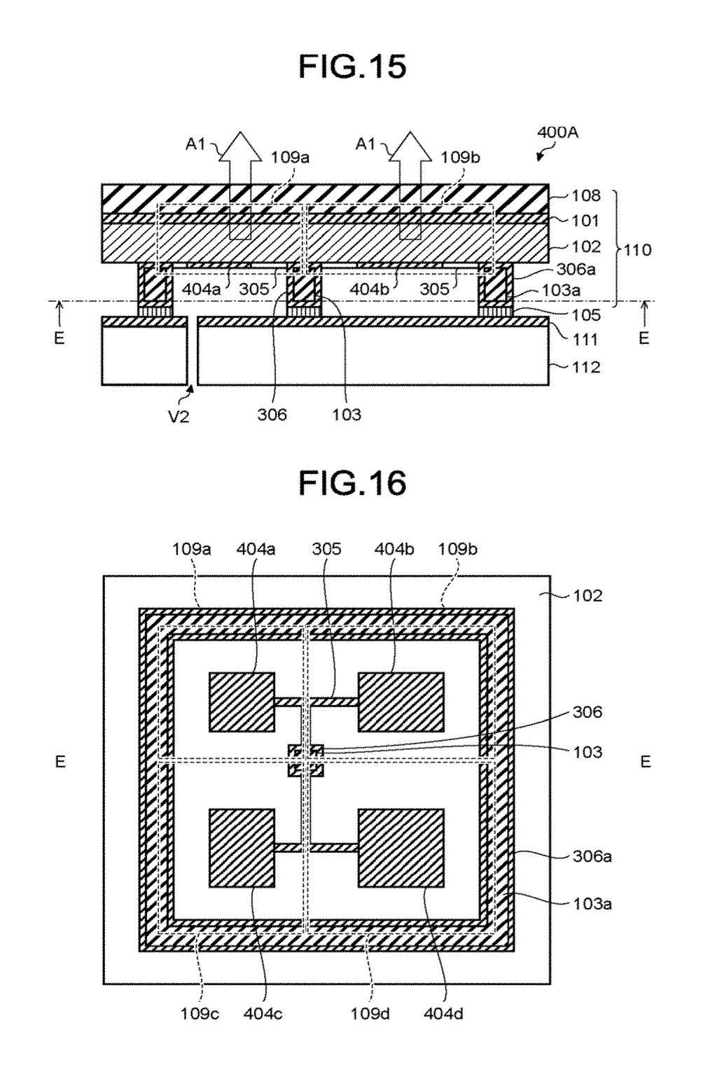

FIG. 15 is a sectional elevation illustrating an example of a schematic configuration of an ultrasonic probe according to the fourth embodiment, and FIG. 16 is a cross-sectional view taken along the line E-E of the ultrasonic probe illustrated in FIG. 15. FIG. 15 illustrates a cross section structure of a plane perpendicular to the pMUT mounting surface of the circuit board 112. In this description, an ultrasonic probe 400A is assumed to include a plurality of elements. In this description, illustrated is the case that each element includes four pMUT elements in two rows and two columns. However, as with those of the foregoing, as long as the diaphragms a no configured in a periodic array, the number of pMUT elements may be changed as appropriate.

As illustrated in FIGS. 15 and 16, in the ultrasonic probe 400A in the fourth embodiment, in the same configuration as that of the ultrasonic probe 300A illustrated in FIGS. 13 and 14, the supporting member 103 surrounded by the supporting member 103a is provided not in substantially the middle of the element but at a location off the center. In such a configuration, the resonance frequencies of the pMUT elements of respective diaphragms 109a to 109d deviate from one another.

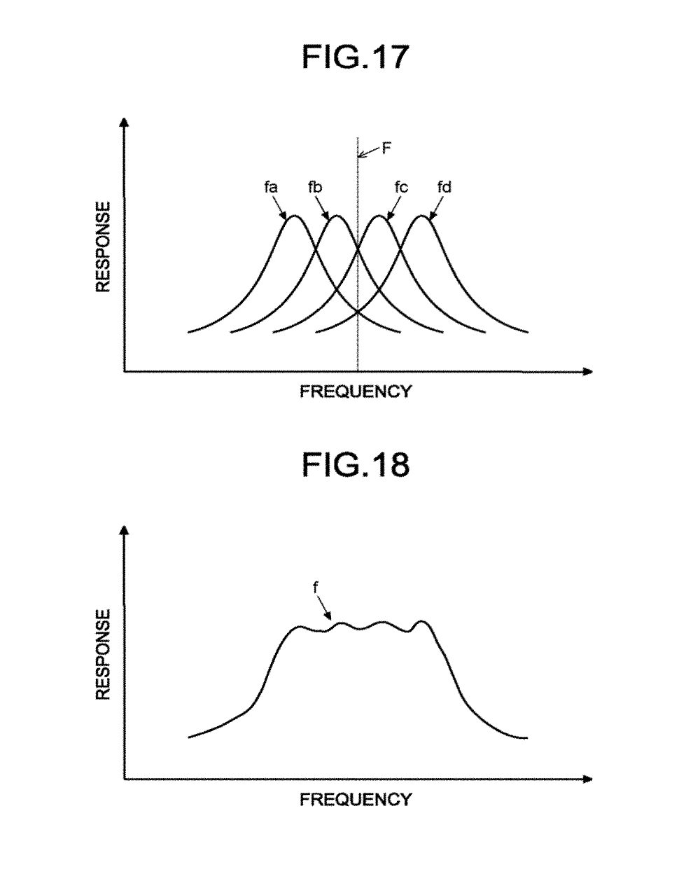

FIG. 17 illustrates frequency spectra of the resonance frequencies of the respective pMUT elements of the ultrasonic probe illustrated in FIGS. 15 and 16. FIG. 18 illustrates a frequency spectrum of the ultrasonic beam for which the frequency spectra illustrated in FIG. 17 were combined, that is, the frequency spectrum output from the ultrasonic probe illustrated in FIGS. 15 and 16. In FIG. 17, a frequency spectrum fa represents the frequency response of the diaphragm 109a, a frequency spectrum fb represents the frequency response of the diaphragms 109b, a frequency spectrum fc represents the frequency response of the diaphragm 109c, and a frequency spectrum fd represents the frequency response of the diaphragm 109d. An F represents the resonance frequency when the supporting member 103 is disposed on the middle, that is, the resonance frequency of the ultrasonic probe 300A illustrated in FIGS. 15 and 16.

As illustrated in FIG. 17, by displacing the location of the supporting member 103 and making the sites of the diaphragms 109a to 109d different from one another, the ultrasound of a different frequency response is output from each pMUT element. The frequency response of the ultrasonic beam output from the ultrasonic probe 400A becomes the one that the ultrasonic beams output from those pMUT elements are combined. Thus, as illustrated in FIG. 18, the ultrasonic beam that has a flattened frequency spectrum f is output from the ultrasonic probe 400A.

Other configurations, operations, and effects can be the same as the configurations, operations, and effects in the above-described embodiments, and thus the redundant explanations are omitted.

### Fifth Embodiment

Next, with reference to the accompanying drawings, the following describes in detail a piezoelectric device and an ultrasonic apparatus according to a fifth embodiment. In the above-described embodiments, as the configuration to decouple the mechanical coupling between the elements, the supporting member 103a that physically fixes the peripheral portion of each element has been provided. In contrast, in the fifth embodiment, the case of decoupling the mechanical coupling between the elements in a configuration different from the above-described embodiments will be described with an example given. In the following description, the configurations the same as those described in the foregoing embodiments are given the identical reference signs and the redundant explanations thereof are omitted.

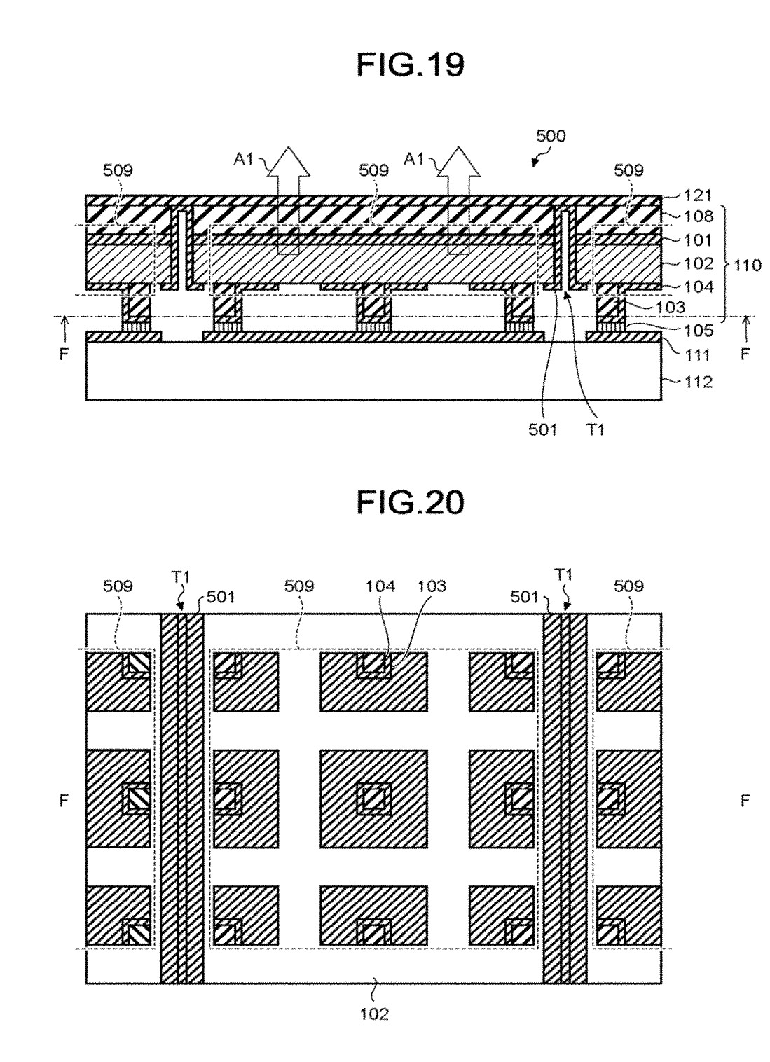

FIG. 19 is a sectional elevation illustrating an example of a schematic configuration of the piezoelectric device in the fifth embodiment, and FIG. 20 is a sectional elevation taken along the line F-F of the piezoelectric device illustrated in FIG. 19. FIG. 19 illustrates a cross section structure of a plane perpendicular to the pMUT mounting surface of the circuit board 112.

As illustrated in FIGS. 19 and 20, a piezoelectric device 500 in the fifth embodiment is provided with a configuration that, in the same configuration as that of the piezoelectric device 100 in the first embodiment, a trench T1 is formed between adjacent elements 509. The trench T1 reaches the silicon thin film 108 via the piezoelectric thin film 102 and the first electrode 101. In the example illustrated in FIG. 19, the trench T1 is provided so as to run through a layered body composed of the piezoelectric thin film 102, the first electrode 101, and the silicon thin film 108.

Furthermore, in the piezoelectric device 500, the buried oxide film 121 of the SOI substrate 120 that was used in the manufacturing process in order to maintain the array of the elements 509 separated by the trench T1 is left. This buried oxide film 121 can also serve as a protective film for preventing the acoustic coupling material and the like from invading into the element 509. The buried oxide film 121 is what is called a thermally oxidized film, and thus it can adequately serve as a protective film even though it is a relatively thin film in the SOI substrate 120.

Moreover, the piezoelectric device 500 includes in-trench wiring 501 provided in the trench T1 in order to electrically connect the first electrodes 101 that were separated for each element 509 by the trench T1. The in-trench wiring 501 in provided from the lateral surface on one side in the trench T1 to the lateral surface on the other side via a bottom surface (the surface of the buried oxide film 121 exposed in the trench T1), so as to electrically connect at least from the first electrode 101 exposed on the lateral surface on one side in the trench T1 to the first electrode 101 exposed on the lateral surface on the other side. Additionally, by making the silicon thin film 108 low in resistivity by doping, the in-trench wiring 501 is also connected via the lateral surface of the silicon thin film 108 in the trench T1, and thus the electrical connection can be further assured.

In the manufacturing method of the piezoelectric device 500 in the fifth embodiment, after patterning the second electrodes 104 from the configuration illustrated in FIG. 5 described in the first embodiment, by using photolithographic and etching techniques, the trench T1 that runs through a layered body composed of the piezoelectric thin film 102, the first electrode 101, and the silicon thin film 108 is formed. For engraving the trench T1, it is possible to use dry etching such as RIE, and thus the trench T1 can be manufactured relatively easily. At that time, it is preferable that at least the silicon thin film 108 be etched, under the condition that the buried oxide film 121 can function as an etching stopper, by appropriately selecting the etching gas used and the like, for example.

The silicon substrate 122 of the SOI substrate 120 after bonding to the circuit board 112 can be removed by using CMP, wet etching for silicon, and others, for example.

As in the foregoing, according to the fifth embodiment, the mechanical coupling between the elements 509 can be further reduced. Accordingly, as the acoustic coupling between the elements 509 is reduced, it is possible to achieve the piezoelectric device 500 for which the acoustic cross-talk is improved.

Other configurations, operations, and effects are the same as those in the above-described embodiments, and thus the redundant explanations are omitted.

### Sixth Embodiment

Next, with reference to the accompanying drawings, the following describes in detail a piezoelectric device and an ultrasonic apparatus according to a sixth embodiment. In the above-described fifth embodiment, as the configuration for which the separated first electrodes 101 are electrically connected one another, the in-trench wiring 501 provided in the trench T1 has been used. In contrast, in the sixth embodiment, another example of the configuration for which the first electrodes 101 separated by the forming of the trench T1 are electrically connected to will be described. In the following description, the configurations the same as those described in the foregoing embodiments are given, the identical reference signs and the redundant explanations thereof are omitted.

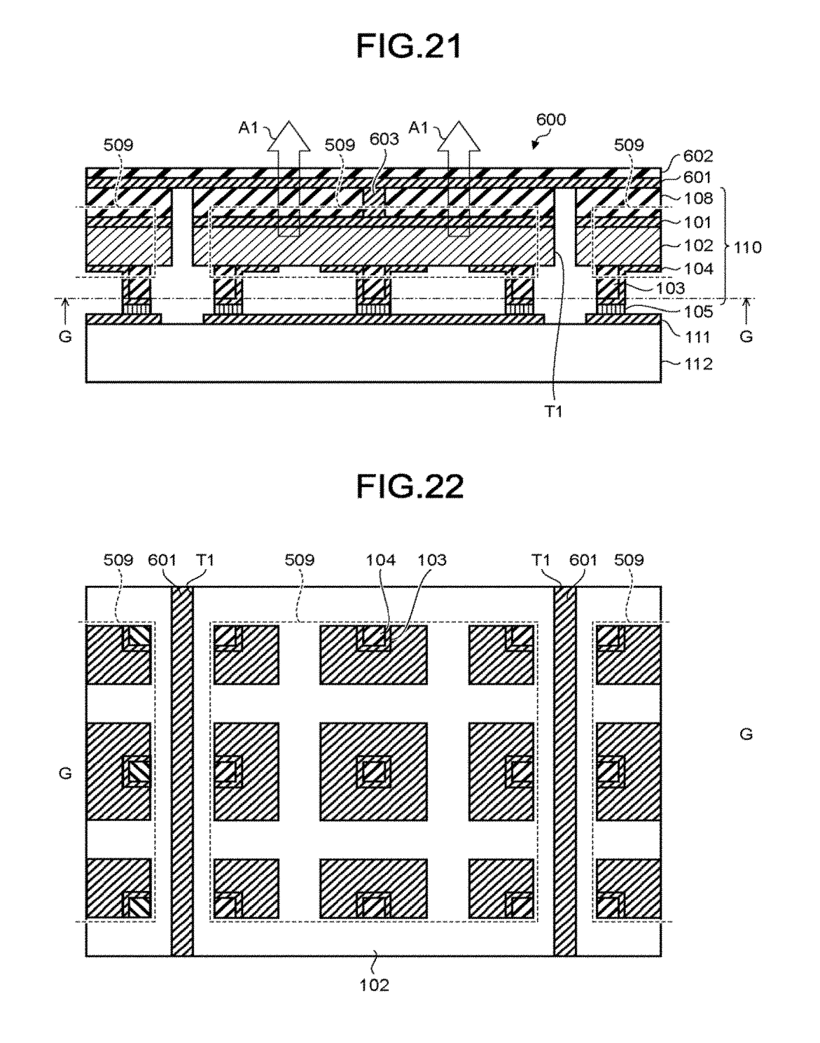

FIG. 21 is a sectional elevation illustrating an example of a schematic configuration of the piezoelectric device in the sixth embodiment, and FIG. 22 is a cross-sectional view taken along the line G-G of the piezoelectric device illustrated in FIG. 21. FIG. 21 illustrates a cross section structure of a plane perpendicular to the pMUT mounting surface of the circuit board 112.

As illustrated in FIGS. 21 and 22, a piezoelectric device 600 in the sixth embodiment is provided with a configuration that, in the same configuration as that of the piezoelectric device 500 in the fifth embodiment, the buried oxide film 121 has been removed. In place of that, the piezoelectric device 600 includes, as a configuration that maintains the array of the elements 509 separated by the trench T1 and that electrically connects the first electrodes 101 separated for each element 509 by the trench T1, a resin sheet 602, a conductive film 601, and a wiring layer 603 that runs through the silicon thin film 108.

The conductive flint 601 is a conductive film of metal or alloy such as gold (Au), silver (Ag), and copper (Cu). This conductive film 601 is provided extending over a plurality of individualized silicon thin films 108 so as to straddle a plurality of elements 509, as with the first electrode 101 before being separated, for example.

The wiring layer 603 is a layer for electrically connecting the first electrode 101 of the individual element 509 to the conductive film 601, and a metal or an alloy such as aluminum (Al), silver (Ag), gold (Au), titanium (Ti), tungsten (W), and nickel (Ni) can be used, for example. In the example illustrated in FIG. 21, the wiring layer 603 is provided so as to run through the silicon thin film 108. However, it is not limited to this configuration, and the wiring layer 603 may be disposed on the lateral surface of the silicon thin film 108 in the trench T1.

The resin sheet 602 is a sheet formed by using thermoplastic resin such as phenol resin and epoxy resin and other various types of resin, and is formed so as to cover the conductive film 601 on the silicon film 108. This resin sheet 6032 can also serve as a protective film for preventing the acoustic coupling material and the like from invading into the element 509.

In the manufacturing method of the piezoelectric device 600 in the sixth embodiment, the processes described by using FIGS. 4 to 6 in the first embodiment are first performed. However, in the process described by using FIG. 4, before forming the first electrode 101 on the silicon thin film 108 of the SOI substrate 120, a process of forming the wiring layer 603 to the silicon thin film 108 is performed.

When the piezoelectric device 100 in the layer structure illustrated in FIG. 1 is manufactured by going through the processes illustrated in FIGS. 4 to 6, by dicing the silicon thin film 108 and the first electrode 101 at certain locations, a plurality of elements 509 are individualized. Then, the resin sheet 602 for which the conductive film 601 is formed on the surface on one side is stuck on the silicon thin film 100 so as to straddle the elements 509. At that time, a pressure applying process and a conductive adhesive may be used such that the electrical connection of the conductive film 601 and the wiring layer 603 is ensured. Accordingly, the piezoelectric device 600 of a layer structure illustrated in FIG. 21 is manufactured.

As in the foregoing, according to the sixth embodiment, because the elements 509 are individualized in the process performed after having bonded the SOI substrate 120 on which the pMUT element array 110 is formed and the circuit board 112, the manufacturing process can be facilitated. Furthermore, because the resin sheet 602 is used as a protective layer for the acoustic coupling material and others, it is possible to achieve the piezoelectric device 600 of higher durability. Moreover, by making the silicon thin film 108 low in resistivity by doping, the wiring layer 603 can be omitted, and thus it is further possible to facilitate the manufacturing process.

Other configurations, operations, and effects are the same as those in the above-described embodiments, and thus the redundant explanations are omitted.

### Seventh Embodiment

Next, with reference to the accompanying drawings, the following describes in detail a piezoelectric device and an ultrasonic apparatus according to a seventh embodiment. In the seventh embodiment, an ultrasonic apparatus (including an ultrasonic probe) using the piezoelectric device in the above-described embodiments will be described in detail with reference to the accompanying drawings. In the following description, the case that the piezoelectric device 100 in the first embodiment, is used is exemplified. However, it is not limited to this, and it is also possible to use the piezoelectric devices in the other embodiments. In the following description, the configurations the same as those described in the foregoing embodiments are given the identical reference signs and the redundant explanations thereof are omitted.

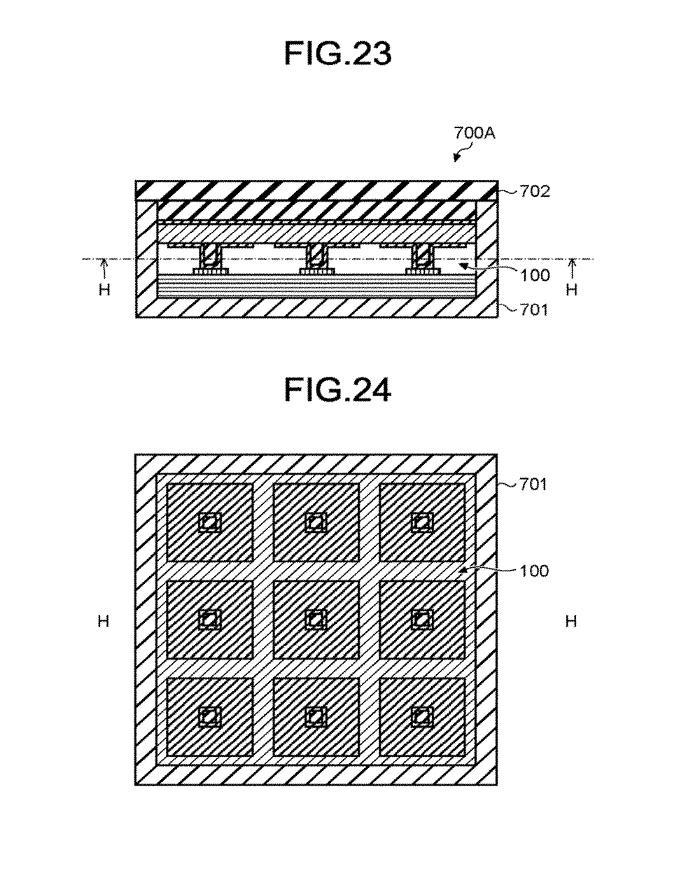

FIG. 23 is a sectional elevation illustrating an example of a schematic configuration of the ultrasonic apparatus in the seventh embodiment, and FIG. 24 is a cross-sectional view taken along the line H-H of the ultrasonic apparatus illustrated in FIG. 23. FIG. 23 illustrates a cross section structure of a plane perpendicular to the pMUT mounting surface of the circuit board 112.

As illustrated in FIGS. 23 and 24, an ultrasonic apparatus 700A in the seventh embodiment includes a housing case 701 that houses therein the piezoelectric device 100 that, is individualized for each element 509, and a protective film 702 that seals the housing case 701. The piezoelectric device 100 is housed in the housing case 701 such that the opposite side of the output surface of the ultrasonic beam, that is, the circuit board 112 side is on the bottom side of the housing case 701.

For the housing case 701, a housing made of plastic or ceramic can be used, for example. The protective film 702 is changeable as appropriate by the use condition, the type of the subject that is an object of application, and others. However, it is desirable that the protective film 702 can achieve matching of acoustic impedance with the subject and also have a function such as waterproof property.

The silicon thin film 108 of the piezoelectric device 100 is fixed onto the protective film 702 by using, for example, adhesive. Meanwhile, the piezoelectric device 100 and the housing case 701 may be fixed or may be not fixed. In the housing case 701, an air vent for the flow of gas with the outside may be provided.

In such a configuration, even once a plurality of ultrasonic apparatuses 700A are adjacently used, because the housing case 701 functions as a partition wall between the adjacent ultrasonic apparatuses 700A, that is, between the elements, the mechanical coupling between the ultrasonic apparatuses 700A is decoupled. Accordingly, because the acoustic coupling between the elements 509 is reduced, it is possible to improve the acoustic cross-talk.

Other configurations, operations, and effects are the same as those in the above-described embodiments, and thus the redundant explanations are omitted.

### Eighth Embodiment

Next, with reference to the accompanying drawings, the following describes in detail a piezoelectric device and an ultrasonic apparatus according to an eighth embodiment. In the eighth embodiment, a modification of the ultrasonic apparatus 700A in the above-described seventh, embodiment will be described with an example given. In the following description, the configurations the same as those described in the foregoing embodiments are given the identical reference signs and the redundant explanations thereof are omitted.

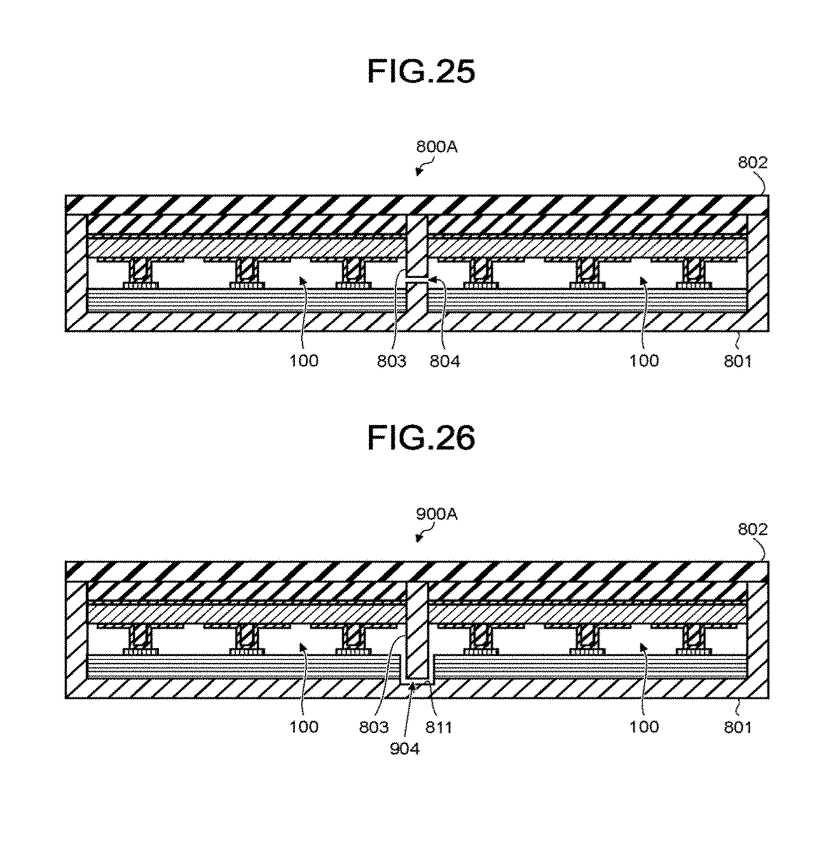

FIG. 25 is a sectional elevation illustrating an example of a schematic configuration of the ultrasonic apparatus in the eighth embodiment. FIG. 25 illustrates a cross section structure of a plane perpendicular to the pMUT mounting surface of the circuit board 112.

As illustrated in FIG. 25, an ultrasonic apparatus 800A in the eighth embodiment is provided with a configuration that a plurality of (two in FIG. 25) ultrasonic apparatuses 700A in the seventh embodiment are concatenated. Specifically, the ultrasonic apparatus 800A is provided with a configuration that a housing case 801 for which the inside is divided into a plurality of housing spaces by a partition 803 is included and that the piezoelectric device 100 is housed in each housing space. The housing spaces of the housing case 801 may be provided with individual protective films (for example, see the protective film 702 in FIG. 23), or may be provided with a protective film 802 in common (see FIG. 25). The protective film 802 can be structured by using the same material as that of the protective film 702 in the seventh embodiment.

The partition 803 of the housing case 801 is provided with a communication path 804 that enables the flow of gas between the adjacent housing spaces. By such a configuration, because the atmospheric pressure changes in the housing space can. be reduced even when the piezoelectric thin film 102 deforms in a barrel shape, it can be reduced that the deformation of the piezoelectric thin film 102 is suppressed. As a result, the piezoelectric thin film 102 can be made to deform efficiently, and efficient generation of the ultrasonic beam is made possible.

By the configuration that the communication path 804 is disposed on the housing case 801 that is relatively easy to work on, because there is no need to form on the circuit board 112 and others a communication path to make the gas flow, it has an advantage of making it possible to facilitate the manufacturing process.

Other configurations, operations, and effects can be the same as the configurations, operations, and effects in the above-described embodiments, and thus the redundant explanations are omitted.

### Ninth Embodiment

Next, with reference to the accompanying drawings, the following describes in detail a piezoelectric device and an ultrasonic apparatus according to a ninth embodiment. In the ninth embodiment, another modification of the ultrasonic apparatus 700A in the above-described seventh embodiment will be described with an example given. In the following description, the configurations the same as those described in the foregoing embodiments are given the identical reference signs and the redundant explanations thereof are omitted.

FIG. 26 is a sectional elevation illustrating an example of a schematic configuration of the ultrasonic apparatus in the ninth embodiment. FIG. 26 illustrates a cross section structure of a plane perpendicular to the pMUT mounting surface of the circuit board 112.

As illustrated in FIG. 26, an ultrasonic apparatus 900A in the ninth embodiment is provided with the same configuration as that of the ultrasonic apparatuses 800A in the eighth embodiment (see FIG. 25). However, in the ultrasonic apparatus 900A, a communication path 904 that enables the flow of gas between the adjacent housing spaces is formed by providing a trench 811 in the housing case 801. That is, an interspace between the trench 811 formed in the bottom portion of the housing case 801 and the partition 803 becomes the communication path 904.

By such a configuration, as with that of the eighth embodiment, because the atmospheric pressure changes in the housing space can be reduced even when the piezoelectric thin film 102 deforms in a barrel shape, it can be reduced that the deformation of the piezoelectric thin film 102 is suppressed. As a result, the piezoelectric thin film 102 can be made to deform efficiently, and efficient generation of the ultrasonic beam is made possible.

By the configuration that the communication path 904 is provided on the housing case 801 that is relatively easy to work on, because there is no need to form on the circuit board 112 and others a communication path to make the gas flow, it has an advantage of mating it possible to facilitate the manufacturing process.

Other configurations, operations, and effects can be the same as the configurations, operations, and effects in the first embodiment, and thus the redundant explanations are omitted.

Other configurations, operations, and effects can be the same as the configurations, operations, and effects in the above-described embodiments, and thus the redundant explanations are omitted.

### Tenth Embodiment

Next, with reference to the accompanying drawings, the following describes in detail a piezoelectric device and an ultrasonic apparatus according to a tenth embodiment. In the tenth embodiment, as a modification of the ultrasonic apparatus 700A in the above-described seventh embodiment, an ultrasonic probe that uses the piezoelectric device as a transmitter of ultrasound will be described as an example. In the following description, the configurations the same as those described in the foregoing embodiments are given the identical reference signs and the redundant explanations thereof are omitted. In the following description, it is described on the basis of the ultrasonic apparatus 800A in the eighth embodiment. However, it is not limited to this, and it is applicable in the same manner to also the ultrasonic apparatus using the piezoelectric device in the ninth embodiment or the other embodiments.

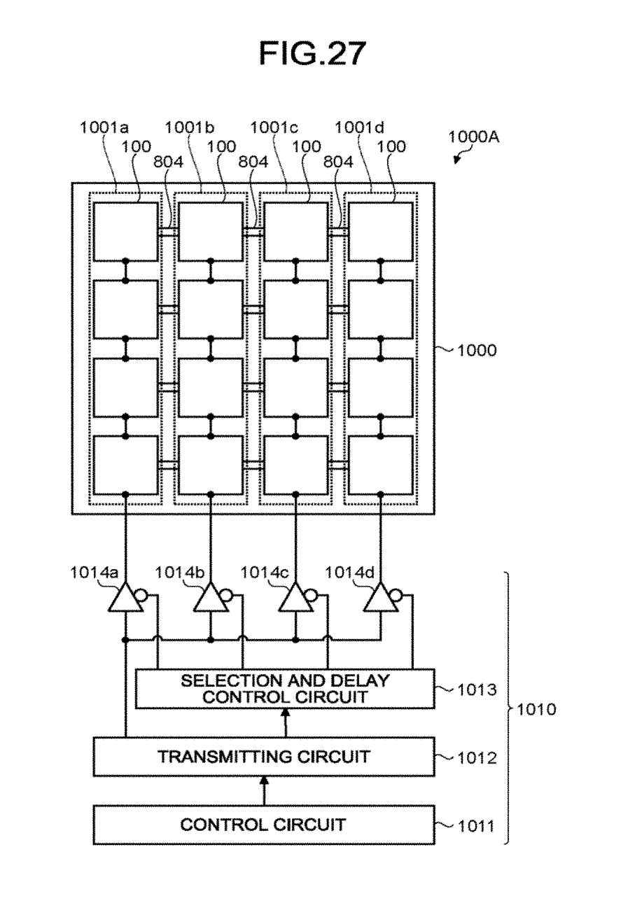

FIG. 27 is a block diagram illustrating an example of a schematic configuration of the ultrasonic probe in the tenth embodiment. As illustrated in FIG. 27, an ultrasonic probe 1000A, includes a piezoelectric device array 1000 that includes a plurality of piezoelectric devices 100, and a transmitting unit 1010 that is mounted on the circuit board 112.

In the tenth embodiment, the piezoelectric device array 1000 includes a total of 16 piezoelectric devices 100 of the first embodiment, in four rows and four columns, for example. The 16 piezoelectric devices 100 are individually housed in the housing space of the housing case 801 that is divided in four rows and four columns with the partitions 803, as illustrated in the eighth embodiment, for example.

The 16 piezoelectric devices 100 of the piezoelectric device array 1000 are grouped into a plurality of driving groups 1001a to 1001d. In the example illustrated in FIG. 27, the 16 piezoelectric devices 100 are grouped such that four piezoelectric devices 100 arrayed in a certain direction (a longitudinal direction in FIG. 27) constitute a single group. It is assumed that each of the driving groups 1001a to 1001d is a driving unit of a higher level than the element that includes a plurality of pMUT elements, and that the piezoelectric devices 100 included in the respective driving groups 1001a to 1001d are driven at the same timing.

Meanwhile, the transmitting unit 1010 on the circuit board 112 side includes a control circuit 1011, a transmitting circuit 1012, a selection and delay control circuit 1013, and driver circuits 1014a to 1014d. The number of the driver circuits 1014a to 1014d can be the same as the number of driving groups 1001a to 1001d, for example.

The control circuit 1011 is composed of an information processor such as a central processing unit (CPU) and a micro processing unit (MPU), and controls the transmitting circuit 1012 in accordance with instructions from the outside.

The transmitting circuit 1012 is what is called a waveform generator circuit, and in accordance with commands from the control circuit 1011, generates a waveform signal to drive the driver circuits 1014a to 1014d.

Each of the driver circuits 1014a to 1014d is electrically connected to the first electrode 101 and/or the second electrodes 104 of the piezoelectric device 100 included in a driving group associated with itself out of the driving groups 1001a to 1001d. Each of the driver circuits 1014a to 1014d modulates the waveform signal input from the transmitting circuit 1012 into a voltage signal to drive the piezoelectric device 100, and inputs a voltage waveform generated thereby into the first electrode 101 and/or the second electrodes 104 of the piezoelectric device 100.

The selection and delay control circuit 1013 is connected to respective enable terminals of the driver circuits 1014a to 1014d, for example. The selection and delay control circuit 1013 selects a driver circuit, to be non-driving, in accordance with instruct ion signals input from the transmitting circuit 1012, out of the driver circuits 1014a to 1014d, and inputs an enable signal into the selected driver circuit. Each of the driver circuits 1014a to 1014d stops the output of the voltage waveform, for ultrasound generation, until the input of the enable signal from the selection and delay control circuit 1013 is stopped.

The control circuit 1011 can control the transmitting circuit 1012 so that each of the driving groups 1001a to 1001d starts oscillating in sequence at a certain delay time interval. In that case, the transmitting circuit 1012, in a stare of outputting a voltage signal to the respective driver circuits 1014a to 1014d, stops the enable signal, which the selection and delay control circuit 1013 inputs into the respective driver circuits 1014a to 1014d, at a certain delay time interval. Accordingly, from the respective driver circuits 1014a to 1014d, the voltage waveform for ultrasound generation is output in sequence at a certain time interval.

In such a configuration and operation, the housing space that houses the piezoelectric device 100 that belongs to one driving group is connected to the housing spaces that house the piezoelectric devices 100 that belong to the other driving groups such that the flow of gas is possible via the communication paths 804. In the example illustrated in FIG. 27, the housing spaces of the adjacent piezoelectric devices 100 between the driving groups are connected via the communication path 804.

As in the foregoing, by spatially connecting the housing spaces that house the piezoelectric devices 100 that are not started driving at the same time in the case of sequential driving, in other words, by spatially connecting the housing spaces that house the piezoelectric devices 100 that belong to different driving groups, it makes it possible to suppress the fluctuation in pressure inside the housing space at the time of start driving. Accordingly, the fact that the deformation of the piezoelectric thin film 102 is hindered by the pressure in the housing space can be reduced. As a result, it makes the efficient generation of the ultrasonic beans possible.

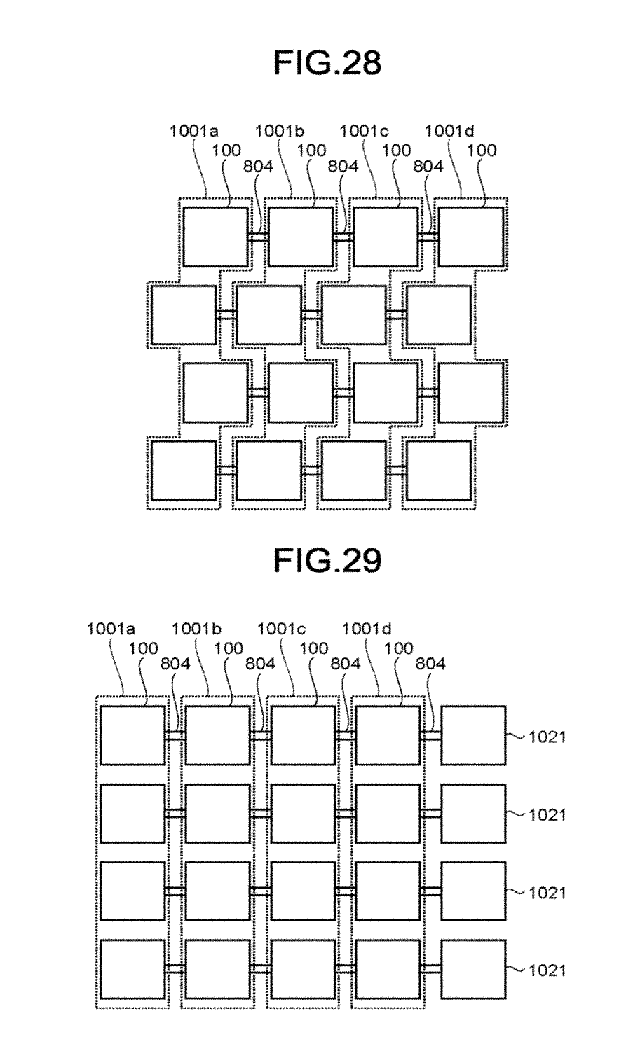

In the tenth embodiment, the case that the piezoelectric devices 100 are arrayed in a matrix form has been exemplified. However, it is not limited to this configuration. For example, as illustrated in FIG. 28, even when a plurality of piezoelectric devices 100 belonging to one group are arrayed by shifting alternately to the left and right and arrayed in a hound's-tooth check form as a whole, spatially connecting the housing spaces that house the piezoelectric devices 100 that are not started driving at the same time in the case of sequential driving makes it possible to suppress the fluctuation in the pressure inside the housing space at the time of start driving. Accordingly, the fact that the deformation, of the piezoelectric thin film 102 is hindered by the pressure in the housing space can be reduced, and as a result, the efficient generation of the ultrasonic beam becomes possible.

Furthermore, in sequential driving, the gas pushed out from the housing space that houses a previously driven piezoelectric device 100 is ultimately accumulated in the housing space that houses the piezoelectric device 100 driven last, and as a result, the air pressure in the housing space that houses the piezoelectric device 100 driven last is increased and that hinders the deformation of the piezoelectric thin film 102 of the piezoelectric device 100. In order to prevent this hindrance, the housing space that houses the piezoelectric device 100 driven last may be provided with an exhaust vent.

Alternatively, as Illustrated in FIG. 29, a dummy housing space (dummy space) 1021 that communicates with the housing space that houses the piezoelectric device 100 driven last via the communication path 604 may be provided, for example. Accordingly, the gas pushed out from the housing space that houses the previously driven piezoelectric device 100 can be prevented from being accumulated in the housing space that houses the piezoelectric device 100 driven last.

Other configurations, operations, and effects can be the same as the configurations, operations, and effects in the above-described embodiments, and thus the redundant explanations are omitted.

### Eleventh Embodiment

Next, with reference to the accompanying drawings, the following describes in detail a piezoelectric device and an ultrasonic apparatus according to an eleventh embodiment. In the eleventh embodiment, as a modification of the ultrasonic apparatus 700A in the above-described seventh embodiment, an ultrasonic diagnostic apparatus that uses the piezoelectric device as an ultrasonic transmitter and receiver will be described as an example. In the following description, the configurations the same as those described in the foregoing embodiments are given the identical reference signs and the redundant explanations thereof are omitted.

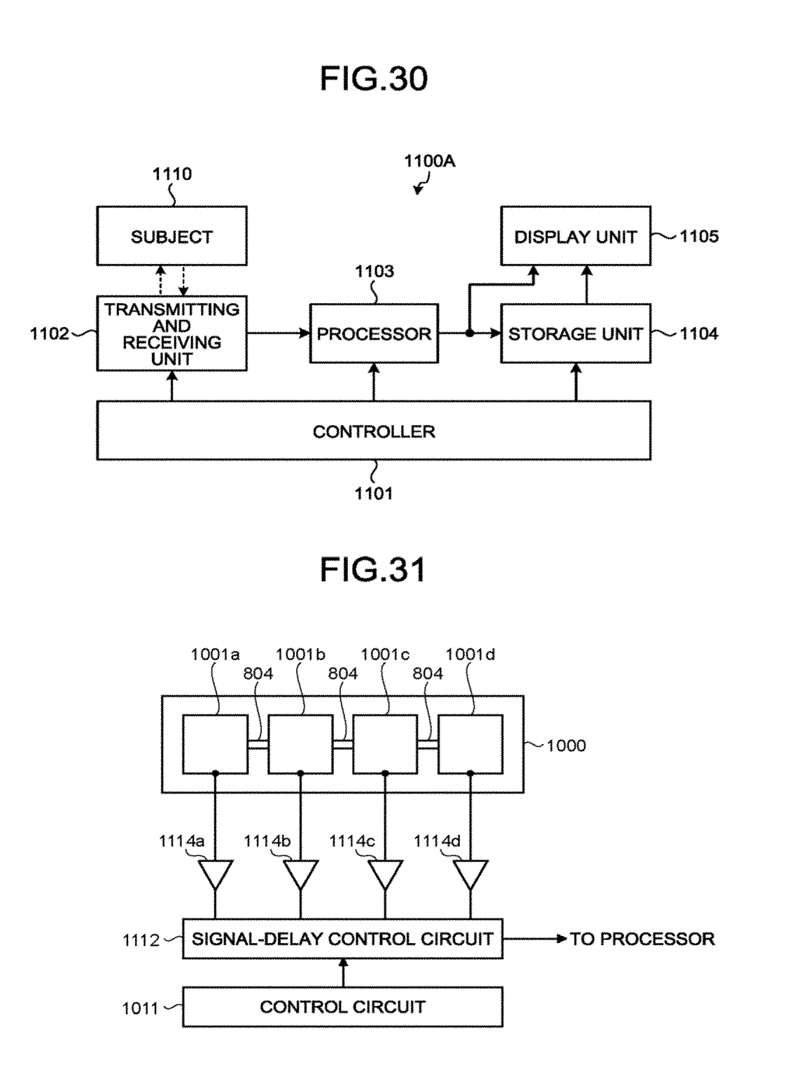

FIG. 30 is a block diagram illustrating an example of a schematic configuration of the ultrasonic diagnostic apparatus in the eleventh embodiment. As illustrated in FIG. 30, an ultrasonic diagnostic apparatus 1100A includes a controller 1101, a transmitting and receiving unit 1102, a processor 1103, a storage unit 1104, and a display unit 1105.

In this configuration, the transmitting and receiving unit 1102 includes the piezoelectric device array 1000 and the transmitting unit 1010 described in the tenth embodiment, for example. As illustrated in FIG. 31, the transmitting and receiving unit 1102 further includes, as a configuration for receiving, pre-amplifiers 1114a to 1114d provided for the respective driving groups 1001a to 1001d, a signal-delay control circuit 1112, and the control circuit 1011. The control circuit 1011 may be identical to the control circuit 1011 in the transmitting unit 1010.

Each of the pre-amplifiers 1114a to 1114d is electrically connected to the first electrode 101 and/or the second electrodes 104 of the piezoelectric device 100 included in the driving group associated with itself out of the driving groups 1001a to 1001d. Each of the pre-amplifiers 1114a to 1114d amplifies an electrical signal changed from the ultrasound by the piezoelectric devices 100 to which it is connected.

The signal-delay control circuit 1112 controls the timing of receiving the electrical signals input via the respective pre-amplifiers 1114a to 1114d. The time difference in the timing that the signal-delay control circuit 1112 receives the electrical signals from the respective pre-amplifiers 1114a to 1114d may be the same as the delay time that the selection and delay control circuit 1013 in the transmitting unit 1010 gives to the respective driver circuits 1014a to 1014d, for example. The electrical signals received by the signal-delay control circuit 1112 are input into the processor 1103 in FIG. 10, for example.

At the time of ultrasonic diagnostic on a subject 1110, the controller 1101 transmits an ultrasonic signal from the transmitting and receiving unit 1102 toward the subject 1110. The transmitted ultrasonic signal is reflected at a certain region of the subject 1110. The transmitting and receiving unit 1102 inputs the ultrasonic signal reflected at the subject 1110, converts the input ultrasonic signal into an electrical signal, and inputs it into the processor 1103.

The processor 1103 generates an ultrasonic image by analyzing the input electrical signal and performing image processing. The generated ultrasonic image may be displayed on the display unit 1105 in real time, or may be displayed on the display unit 1105 as needed after storing once in the storage unit 1104.

As in the foregoing, the piezoelectric device exemplified in the above-described eleventh embodiment can be applied to also the ultrasonic diagnostic apparatus that uses the piezoelectric device as an ultrasonic transmitter and receiver.

Other configurations, operations, and effects are the same as those in the above-described embodiments, and thus the redundant explanations are omitted.

### Twelfth Embodiment

In a twelfth embodiment, an example of a configuration of the pMUT element in the above-described embodiments will be described specifically. In the following description, it is described by referring to the pMUT elements that the piezoelectric device 300 in the third embodiment includes. However, it is also possible to be applied to the pMUT elements of the other embodiments in the same manner. In the following description, the configurations the same as those described in the foregoing embodiments are given the identical reference signs and the redundant explanations thereof are omitted.

First, the composition ratios among the diaphragm 109, the second electrode 304, and the supporting members 103 will be described. In the twelfth embodiment, in the configuration of the piezoelectric device 300 illustrated in FIG. 10, when the thickness of the piezoelectric thin film 102 is defined as hₘ and the thickness of the silicon thin film 108 is defined as hₚ, the ratio κ of the thickness of the piezoelectric thin film 102 to the thickness (hₘ+hₚ) of the layered body (the piezoelectric thin film 102 and the silicon thin film 108) that deforms in a barrel shape at the time of generating ultrasound is expressed by the following Expression (1).

$$κ = \frac{{h}_{m}}{{h}_{m} + {h}_{p}} \qquad ( 1 )$$

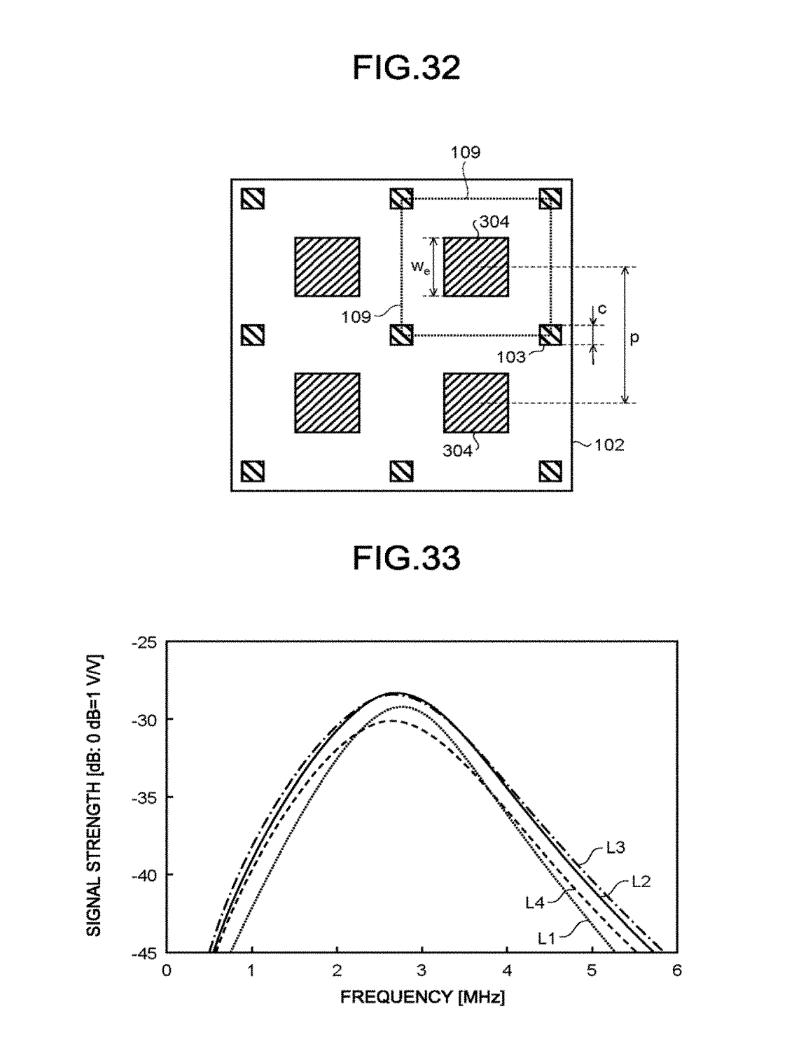

As illustrated in FIG. 32, when the width of the second electrode 304 formed on the second surface of the piezoelectric thin film 102 is defined as wₑ and the distance between the canters of the supporting members 103, that is, the pitch of the diaphragms 109, is defined as p, the ratio ξ thereof is expressed as the following Expression 2. In FIG. 32, the c represents a thickness of the supporting member 103. In this description, it is assumed that the cross-sections of the diaphragm 109, the second electrode 304, and the supporting member 103 are all in a square shape.

$$ξ = \frac{{w}_{e}}{P} \qquad ( 2 )$$

The transmitting and receiving sensitivity characteristics of the piezoelectric device 300 when the ratio (c/p) of the thickness c of the supporting member 103 to the pitch p of the diaphragms 109 is varied are simulated. FIG. 33 is a graph illustrating the simulation result. In this simulation, PZT was used as the piezoelectric thin film. 102, the pitch p of the diaphragms 109 was made to be 150 μm, and the ratio κ was made to be 0.5.

In FIG. 33, a line L1 indicates the case that the ratio c/p was made to be 0.2 (this is defined as a first example), a line L2 indicates the case that the ratio c/p was made to be 0.3 (this is defined as a second example), a line L3 indicates the case that, the ratio c/p was made to be 0.4 (this is defined as a third example), and a line L4 indicates the case that the ratio c/p was made to be 0.5 (this is defined as a fourth example). In the respective cases, as a result of obtaining the ratio ξ to be optimal, the ratio ξ in the case of the first example was 0.5, the ratio ξ the case of the second example was 0.4, the ratio ξ in the case of the third example was 0.4, and the ratio ξ in the case of the fourth example was 0.35.

As illustrated in FIG. 33, the transmitting and receiving sensitivity characteristics in the second example and the third example indicate better values than those of the other first example and fourth example. This indicates that it is preferable that the ratio c/p be 0.3 to 0.4.

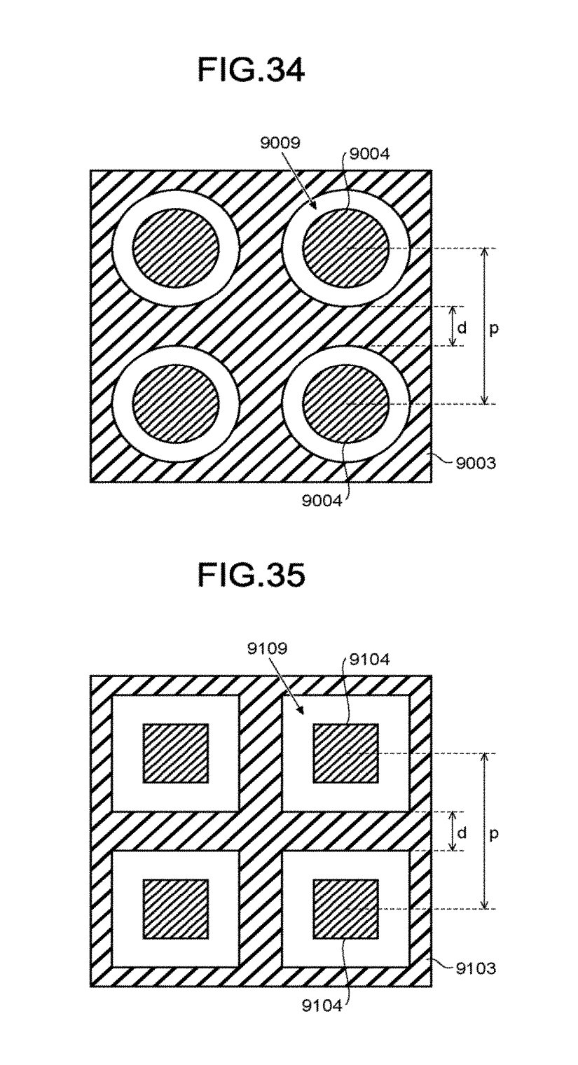

Next, the area usage efficiency will be described. In this description, as comparative examples to the configuration illustrated in FIG. 33, a first comparative example illustrated in FIG. 34 and a second comparative example illustrated in FIG. 35 are given.

In a piezoelectric device according to the first comparative example illustrated in FIG. 34, a diaphragm 9009 equivalent to a single pMUT element is in a columnar shape, and along with that, an electrode 9004 also, which is equivalent to the second electrode 304 in the twelfth embodiment, is in a circular shape. The columnar diaphragms 9009 are sectioned by a partition wall 9003.

Meanwhile, in a piezoelectric device according to the second comparative example illustrated in FIG. 35, a diaphragm 9109 equivalent to a single pMUT element is in a square pillar shape, and along with that, an electrode 9104 also, which is equivalent to the second electrode 304 in the twelfth embodiment, is in a square shape. The square-pillar shaped diaphragms 9109 are sectioned by a partition wall 9103.

An area usage efficiency F0 of the configuration illustrated in FIG. 32 is expressed by the following Expression 3. An area usage efficiency F1 of the first comparative example illustrated in FIG. 34 is expressed by the following Expression 4, and an area usage efficiency F2 of the second comparative example illustrated in FIG. 35 is expressed by the following expression 5.

$$\begin{matrix}F 1 = \frac{π}{4} \frac{{( p - d )}^{2}}{{P}^{2}} = \frac{π}{4} {( 1 - \frac{d}{p} )}^{2} & ( 4 ) \\ F 2 = \frac{{( p - d )}^{2}}{{P}^{2}} = {( 1 - \frac{d}{p} )}^{2} & ( 5 )\end{matrix}$$

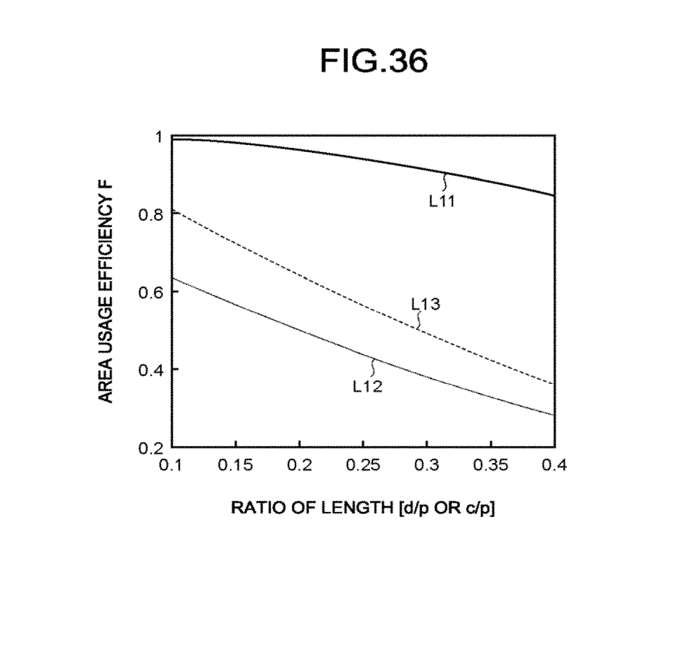

FIG. 36 illustrates the result of area usage efficiencies calculated by using Expression 3 to Expression 5 for the respective configurations illustrated in FIGS. 32, 34, and 35. In FIG. 36, a line L11 indicates the area usage efficiency F0 calculated by using Expression 3 for the configuration illustrated in FIG. 32, a line L12 indicates the area usage efficiency F1 calculated by using Expression 4 for the configuration illustrated in FIG. 34, and a line L13 indicates the area usage efficiency F2 calculated by using Expression 5 for the configuration illustrated in FIG. 33.

As it is apparent by referring to FIG. 36, out of the configurations illustrated in FIGS. 32, 34, and 35, the configuration illustrated in FIG. 32, that is, the configuration of the piezoelectric device 300 described in the third embodiment has the highest area usage efficiency.

From the foregoing, according to the above-described twelfth embodiment, a piezoelectric device and an ultrasonic apparatus that are capable of increasing the area usage efficiency and efficiently generating an ultrasonic beam can be achieved.

Other configurations, operations, and effects can be the same as the configurations, operations, and effects in the above-described embodiments, and thus the redundant explanations are omitted.

The above-described embodiments and modifications are mere examples to implement the present invention, and the invention is not limited thereto. Making various modifications depending on the specifications and such is within the scope of the invention. Furthermore, within the scope of the invention, it is self-evident from the foregoing that various other embodiments are possible. For example, it is obvious that it is also possible to combine the modification accordingly illustrated for one embodiment with the other embodiments.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A piezoelectric device comprising:
a piezoelectric thin film;
a first electrode disposed on a first surface of the piezoelectric thin film;
a substrate provided with an electrode pad;
a plurality of pillar-shaped first supporting members provided between a second surface on an opposite side of the first surface of the piezoelectric thin film and the electrode pad of the substrate so as to fix the piezoelectric thin film onto the substrate; and
a plurality of second electrodes electrically connected to the electrode pad from a part of the second surface of the piezoelectric thin film via a lateral surface of the first supporting member, wherein
the piezoelectric thin film, the first electrode, and the second electrodes compose a plurality of diaphragms each of which is a transducer element,
the first supporting members are provided at locations at which the respective diaphragms are sectioned, and
the first electrode is provided in common to the diaphragms.

2. The piezoelectric device according to claim 1, wherein
the second electrodes are provided corresponding to the first supporting members on a one-to-one basis, and
each second electrode is extending to the electrode pad from the part of the second surface of the piezoelectric thin film via the lateral surface of the first supporting member.

3. The piezoelectric device according to claim 1, further comprising a plurality of auxiliary electrodes extending to the electrode pad from each second electrode via a lateral surface of the first supporting member, wherein
the second electrode is provided on substantially a middle of an area sectioned by the first supporting members on the second surface of the piezoelectric thin film.

4. The piezoelectric device according to claim 1, further comprising:
a second supporting member provided between the second surface of the piezoelectric thin film and the electrode pad of the substrate so as to fix the piezoelectric thin film onto the substrate at a periphery portion of an element constituted by at least two diaphragms out of the diaphragms; and
a plurality of third electrodes electrically connected to the electrode pad from a part of the second surface of the piezoelectric thin film via a lateral surface of the second supporting member, wherein
the second supporting member is provided with a communication path for gas to flow.

5. The piezoelectric device according to claim 1, further comprising:
a second supporting member provided between the second surface of the piezoelectric thin film and the electrode pad of the substrate so as to fix the piezoelectric thin film onto the substrate at as periphery portion of an element constituted by at least two diaphragms out of the diaphragms; and
a plurality of third electrodes electrically connected to the electrode pad from a part of the second surface of the piezoelectric thin film via a lateral surface of the second supporting member, wherein
the substrate is provided with a communication path for gas to flow.

6. The piezoelectric device according to claim 1, wherein the first supporting member is provided at a location at which at least two diaphragms out of the diaphragms have areas along the second surface of the piezoelectric thin film different from one another.

7. The piezoelectric device according to claim 1, further comprising a protective film disposed on the first surface side of the piezoelectric thin film.

8. The piezoelectric device according to claim 1, further comprising a trench running through the piezoelectric thin film and the first electrode so as to divide the piezoelectric thin film and the first electrode into a plurality of pieces, the trench being provided on a periphery portion of an element constituted by at least two diaphragms out of the diaphragms.

9. The piezoelectric device according to claim 8, further comprising in-trench wiring provided on the trench so as to electrically connect a plurality of first electrodes divided by the trench.

10. The piezoelectric device according to claim 8, further comprising:
a conductive film provided to extend over a plurality of piezoelectric thin films divided by the trench; and
wiring electrically connecting a plurality of first electrodes divided by the trench and the conductive film.

11. An ultrasonic apparatus, comprising the piezoelectric device according to claim 1, wherein
the diaphragms are sectioned into a plurality of elements each including at least two diaphragms,
the substrate includes a drive circuit that groups the elements into driving groups of one or more and drives the elements in units of the driving groups,
the piezoelectric device includes a first partition wall sectioning the elements,
the piezoelectric thin film, the substrate, and the first partition wall form a first space for each element, and
the first partition wall is provided with a communication path that makes the first space or an element, which belongs to a first driving group, communicate with the first space of an element that belongs to a second driving group that is different from the first driving group.

12. The ultrasonic apparatus according to claim 11, wherein at least one of the communication paths communicates with outside air.

13. The ultrasonic apparatus according to claim 11, further comprising a second partition wall forming a second space different from the first space together with the piezoelectric thin film and the substrate, wherein
at least one of the communication paths communicates with the second space.
